SEQUENCE LISTING (25 sequences)

SEQ ID NO: 1 - protein, 170 aa, Artificial Sequence
  source: /note="Description of Artificial Sequence: Synthetic polypeptide"
KKGGSHHHHHHSGGMTIIEGQLVANEMKIGIVVSRFNELITSKLLSGAVDGLLRHGVSEEDIDIVWVPGAFEIPYMARKMALYKDYDAIICLGVVIKGSTDHYDYVCNEVTKGIGHLNSQSDIPHIFGVLTTDNIEQAIERAGTKAGNKGYDCALSAIEMVNLDKKLRER

SEQ ID NO: 2 - protein, 155 aa, Streptococcus agalactiae
MKFCETKKILNQLVADLSVFSVRIHQVHWYMRGGRFLTLHPKMDDLMEQINDQLDVISERLITLDGSPYSTLEEFFINSKLKEEKGSWNKTIDEQINYLLEGYSYLISIYEEGIEIAGKEGDDCTEDIFIGSKSELEKEIWMLKAELGNSPELDK

SEQ ID NO: 3 - protein, 169 aa, Artificial Sequence
  source: /note="Description of Artificial Sequence: Synthetic polypeptide"
KKGGSHHHHHHSGGMKFSETKKILNQLVADLSVFSVRIHQVHWYMRGGRFLTLHPKMDDLMEQINDQLDVISERLITLDGSPYSTLEEFFINSKLKEEKGSWNKTIDEQINYLLEGYSYLISIYEEGIEIAGKEGDDSTEDIFIGSKSELEKEIWMLKAELGNSPELDK

SEQ ID NO: 4 - protein, 175 aa, Streptococcus pyogenes
MTNTLVENIYASVTHNISKKEASKNEKTKAVLNQAVADLSVAASIVHQVHWYMRGPGFLYLHPKMDELLDSLNANLDEVSERLITIGGAPYSTLAEFSKHSKLDEAKGTYDKTVAQHLARLVEVYLYLSSLYQVGLDITDEEGDAGTNDLFTAAKTEAEKTIWMLQAERGQGPAL

SEQ ID NO: 5 - protein, 175 aa, Streptococcus pyogenes
MTNTLVENIYASVTHNISKKEASKNEKTKAVLNQAVADLSVAASIVHQVHWYMRGPGFLYLHPKMDELLDSLNANLDEMSERLITIGGAPYSTLAEFSKHSKLDEAKGTYDKTVAQHLARLVEVYLYLSSLYQVGLDITDEEGDAGTNDLFTAAKTEAEKTIWMLQAERGQGPAL

SEQ ID NO: 6 - protein, 155 aa, Streptococcus agalactiae
MKFEKTKEILNQLVADLSQFSVVIHQTHWYMRGPEFLTLHPQMDEYMDQINEQLDVVSERLITLDGSPFSTLREFAENTKIEDEIGNWDRTIPERMEKLVAGYRYLADLYAKGIEVSGEEGDDCTQDIFIANKTDIEKNIWMLQAKLGKAPGIDA

SEQ ID NO: 7 - protein, 155 aa, Artificial Sequence
  source: /note="Description of Artificial Sequence: Synthetic polypeptide"
MKFEKTKEILNQLVADLSQFSVVIHQTHWYMRGPEFLTLHPQMDEYMDQINEQLDVVSERLITLDGSPFSTLREFAENTKIEDEIGNWDRTIPERMEKLVAGYRYLADLYAKGIEVSGEEGDDSTQDIFIANKTDIEKNIWMLQAKLGKAPGIDA

SEQ ID NO: 8 - protein, 153 aa, Streptococcus agalactiae
MVYEKTQAVLNQAVADLSKASSIVHQTHWYMRGPGFFYLHPKMDELMDELNETLDVVSERLITIGGAPYSTLKEFDEHSKLTEEVGSYDKSMTERLEKLADVYAYLSDLFQEGLDVSDEEGDDPSNGIFSDAQDAVQKTLWMLQAEIGRSSGL

SEQ ID NO: 9 - protein, 165 aa, Artificial Sequence
  source: /note="Description of Artificial Sequence: Synthetic polypeptide"
MKFEKTKEILNQLVADLSQFSVVIHQTHWYMRGPEFLTLHPQMDEYMDQINEQLDVVSERLITLDGSPFSTLREFAENTKIEDEIGNWDRTIPERMEKLVAGYRYLADLYAKGIEVSGEEGDDSTQDIFIANKTDIEKNIWMLQAKLGKAPGIDAGSSGHHHHHH

SEQ ID NO: 10 - protein, 163 aa, Artificial Sequence
  source: /note="Description of Artificial Sequence: Synthetic polypeptide"
MVYEKTQAVLNQAVADLSKASSIVHQTHWYMRGPGFFYLHPKMDELMDELNETLDVVSERLITIGGAPYSTLKEFDEHSKLTEEVGSYDKSMTERLEKLADVYAYLSDLFQEGLDVSDEEGDDPSNGIFSDAQDAVQKTLWMLQAEIGRSSGLGSSGHHHHHH

SEQ ID NO: 11 - protein, 175 aa, Artificial Sequence
  source: /note="Description of Artificial Sequence: Synthetic polypeptide"
MTNTLVENIYASVTHNISKKEASKNEKTQAVLNQAVADLSKASSIVHQTHWYMRGPGFFYLHPKMDELMDELNETLDVVSERLITIGGAPYSTLKEFDEHSKLTEEVGSYDKSMTERLEKLADVYAYLSDLFQEGLDVSDEEGDDPSNGIFSDAQDAVQKTLWMLQAEIGRSSGL

SEQ ID NO: 12 - protein, 184 aa, Artificial Sequence
  source: /note="Description of Artificial Sequence: Synthetic polypeptide"
MKKIEKRIEKIEKRIKKIEKRAKFVAAWTLKAAAEKTQAVLNQAVADLSKASSIVHQTHWYMRGPGFFYLHPKMDELMDELNETLDVVSERLITIGGAPYSTLKEFDEHSKLTEEVGSYDKSMTERLEKLADVYAYLSDLFQEGLDVSDEEGDDPSNGIFSDAQDAVQKTLWMLQAEIGRSSGL

SEQ ID NO: 13 - protein, 185 aa, Artificial Sequence
  source: /note="Description of Artificial Sequence: Synthetic polypeptide"
MTNTLVENIYASVTHNISKKEASKNEKTQAVLNQAVADLSKASSIVHQTHWYMRGPGFFYLHPKMDELMDELNETLDVVSERLITIGGAPYSTLKEFDEHSKLTEEVGSYDKSMTERLEKLADVYAYLSDLFQEGLDVSDEEGDDPSNGIFSDAQDAVQKTLWMLQAEIGRSSGLGSSGHHHHHH

SEQ ID NO: 14 - protein, 194 aa, Artificial Sequence
  source: /note="Description of Artificial Sequence: Synthetic polypeptide"
MKKIEKRIEKIEKRIKKIEKRAKFVAAWTLKAAAEKTQAVLNQAVADLSKASSIVHQTHWYMRGPGFFYLHPKMDELMDELNETLDVVSERLITIGGAPYSTLKEFDEHSKLTEEVGSYDKSMTERLEKLADVYAYLSDLFQEGLDVSDEEGDDPSNGIFSDAQDAVQKTLWMLQAEIGRSSGLGSSGHHHHHH

SEQ ID NO: 15 - protein, 25 aa, Streptococcus pyogenes
MTNTLVENIYASVTHNISKKEASKN

SEQ ID NO: 16 - protein, 21 aa, Artificial Sequence
  source: /note="Description of Artificial Sequence: Synthetic peptide"
MKKIEKRIEKIEKRIKKIEKR

SEQ ID NO: 17 - protein, 13 aa, Artificial Sequence
  source: /note="Description of Artificial Sequence: Synthetic peptide"
AKFVAAWTLKAAA

SEQ ID NO: 18 - protein, 177 aa, Artificial Sequence
  source: /note="Description of Artificial Sequence: Synthetic polypeptide"
MTNTLVENIYASVTHNISKKEASKNEKTKEILNQLVADLSQFSVVIHQTHWYMRGPEFLTLHPQMDEYMDQINEQLDVVSERLITLDGSPFSTLREFAENTKIEDEIGNWDRTIPERMEKLVAGYRYLADLYAKGIEVSGEEGDDSTQDIFIANKTDIEKNIWMLQAKLGKAPGIDA

SEQ ID NO: 19 - protein, 10 aa, Artificial Sequence
  source: /note="Description of Artificial Sequence: Synthetic peptide"
GSSGHHHHHH

SEQ ID NO: 20 - protein, 8 aa, Artificial Sequence
  source: /note="Description of Artificial Sequence: Synthetic peptide"
DYKDDDDK

SEQ ID NO: 21 - protein, 9 aa, Artificial Sequence
  source: /note="Description of Artificial Sequence: Synthetic peptide"
AWRHPQFGG

SEQ ID NO: 22 - protein, 8 aa, Artificial Sequence
  source: /note="Description of Artificial Sequence: Synthetic peptide"
WSHPQFEK

SEQ ID NO: 23 - protein, 28 aa, Artificial Sequence
  source: /note="Description of Artificial Sequence: Synthetic peptide"
WSHPQFEKGGGSGGGSGGGSWSHPQFEK

SEQ ID NO: 24 - protein, 155 aa, Artificial Sequence
  source: /note="Description of Artificial Sequence: Synthetic polypeptide"
MKFSETKKILNQLVADLSVFSVRIHQVHWYMRGGRFLTLHPKMDDLMEQINDQLDVISERLITLDGSPYSTLEEFFINSKLKEEKGSWNKTIDEQINYLLEGYSYLISIYEEGIEIAGKEGDDSTEDIFIGSKSELEKEIWMLKAELGNSPELDK

SEQ ID NO: 25 - protein, 6 aa, Artificial Sequence
  source: /note="Description of Artificial Sequence: Synthetic 6xHis tag"
HHHHHH

(19) United States
(12) Patent Application Publication
(10) Pub. No.: US 2023/0303637 A1
(43) Pub. Date: Sep. 28, 2023

(54) GBS FERRITIN NANOPARTICLES

(71) Applicant: GLAXOSMITHKLINE BIOLOGICALS SA, Rixensart (BE)

(72) Inventors: Matthew James BOTTOMLEY, Rockville, MD (US); Newton Muchugu WAHOME, Rockville, MD (US)

(73) Assignee: GLAXOSMITHKLINE BIOLOGICALS SA, Rixensart (BE)

(21) Appl. No.: 18/008,767

(22) PCT Filed: Jun. 11, 2021
(86) PCT No.: PCT/IB2021/055156

§ 371 Date: Dec. 7, 2022

Related U.S. Application Data
(60) Provisional application No. 63/038,225, filed on Jun. 12, 2020.

(51) Int. Cl. C07K 14/315; A61K 39/09

(52) U.S. Cl. CPC C07K 14/315; A61K 39/092; A61K 2039/55555; C07K 2319/21

(57) ABSTRACT

Polypeptides capable of self-assembling into a nanoparticle, and nanoparticles made up of such polypeptides.

### SEQUENCE LISTING

The instant application contains a Sequence Listing which has been submitted electronically in ASCII format and is hereby incorporated by reference in its entirety. Said ASCII copy, created on May 20, 2021, is named VU66891_WO_SL.txt and is 28,111 bytes in size.

### FIELD OF THE INVENTION

This invention relates to polypeptide nanoparticles, and such nanoparticles that display antigenic molecules on their surface.

### BACKGROUND

Ferritin is a ubiquitous, naturally occurring iron transport and storage protein found in both prokaryotes and eukaryotes, including bacteria, insects and mammals. Ferritin functions to protect cells against oxidative damage. Mammalian ferritin is typically composed of 24 polypeptide subunits of ferritin heavy (H) and light (L) chains that self-assemble into a roughly spherical structure. Bacterial ferritin typically has a single polypeptide subunit type. Bacterial and mammalian ferritins appear to have a common evolutionary origin.

A “ferritin-like” protein discovered in stationary phase Escherichia coli and termed “DNA-binding Protein from Starved cells” (DPS) also functions to protect the cell from oxidative damage. Grant et al., Nat Struct Biol. 5:294-303 (1998). Like ferritin, E. coli DPS is a roughly spherical molecule, and is made up of 12 homologous polypeptide subunits.

Naturally-occurring multimeric polypeptides have been proposed for use as drug delivery vehicles and as scaffolds for the display of antigenic molecules. See, e.g., Didi et al., New Biotechnology 32(6):651-657 (2015); Lopez-Sagaseta et al., Computational and Structural Biotech Journal 14:58-68 (2016); Wang et al., Front Chem Sci Eng. 11(4):633-646 (2017).

There is a need for nanoparticle carriers suitable for medical, including vaccine, uses.

### SUMMARY OF THE INVENTION

The present invention provides polypeptides capable of self-assembling into a nanoparticle. Such a polypeptide may have a sequence selected from SEQ ID NO: 4, SEQ ID NO: 5, SEQ ID NO: 2, SEQ ID NO: 6, SEQ ID NO: 7, and SEQ ID NO: 8, or the sequence of a naturally-occurring S. agalactiae protein sequence where one or more naturally-occurring cysteine residues have been substituted with serine. The polypeptide may include an N-terminal amino acid sequence forming an alpha helix.

The present invention further provides polypeptides capable of self-assembling into a nanoparticle, which are joined at the N-terminus to an antigenic molecule.

Also provided are isolated nucleic acid molecules encoding a polypeptide of the invention, recombinant vectors, host cells, and processes of producing such polypeptides.

Also provided are nanoparticles (NP) made up of such polypeptides, and such nanoparticles with antigenic molecules bound to the exterior surface of the NP.

Also provided are immunogenic or pharmaceutical compositions comprising such NPs, and methods of using the same.

### BRIEF DESCRIPTION OF THE FIGURES

FIG. 1 graphs the expected molecular weight profile of nanoparticles made up of SEQ ID NO:13 (GBS 14747 + N-terminal helix + His tag; solid black line), SEQ ID NO:9 (GBS 092 + His Tag; solid gray line), and SEQ ID NO:10 (GBS 14747 + His Tag; dashed black line). The dashed gray line represents molecular weight (MW) markers for standard proteins with peaks at approximately 27 mins for a protein of 670 kDa, and at approximately 36 mins for a protein of 158 kDa. The X-axis is time (minutes)), Y-Axis is Absorbance Units (AU).

FIGS. 2A - 2C graphs the results of Dynamic Light Scattering (DLS) assessment of the size distribution profile of nanoparticles made up of SEQ ID NO:10 (FIG. 2A), SEQ ID NO:9 (FIG. 2B), and SEQ ID NO:13 (FIG. 2C). X-Axis is diameter in nanometers (nm); Y-axis is % Mass.

FIGS. 3A - 3C graphs the results of Nano-Differential Scanning Fluorimetry (Nano-DSF) to determine thermostability of nanoparticles made up of SEQ ID NO:10 (FIG. 3A), SEQ ID NO:9 (FIG. 3B), and SEQ ID NO:13 (FIG. 3C). Samples were run in triplicate. Each image contains two sets of curves. The upper curves are ratiometric measurement of the fluorescent signal against increasing temperature. X-Axis is temperature (°); Y-axis is intrinsic tryptophan fluorescence intensity in arbitrary units (AU). While the lower curves are the first derivatives.

FIGS. 4A and 4B graphically depict the X-ray structure of a nanoparticle made up of SEQ ID NO: 9 subunits (FIG. 4A) and a nanoparticle made up of polypeptide subunits of SEQ ID NO: 13 (FIG. 4B). Asymmetric units are in black, whereas biologically assembled nanoparticles are shown in grey. Metal particles are shown as black spheres.

FIG. 5 depicts the 1.9 Angstrom crystal structure of a dodecameric nanoparticle made up of subunits of SEQ ID NO:9. Exposed lysines are highlighted as sticks.

FIG. 6 graphically represents a computationally-derived model of a nanoparticle of the present invention chemically conjugated to GBS capsular polysaccharide (CPS) antigen, one CPS per protein monomer, to provide a nanoparticle carrying a minimum of 12 copies of the GBS CPS on its outer surface.

FIG. 7A illustrates the process of depolymerization of GBS serotype II capsular polysaccharides to provide shorter oligosaccharide, using N-deAcetylation with NaOH, oxidation with NaNO₂, and N-acetylation, followed by purification using a desalting column.

FIG. 7B illustrates the modification of GBS serotype II short oligosaccharides with a hydrazine linker (ADH) and an active ester spacer (SIDEA).

FIG. 7C illustrates the oxidation of GBS serotype II capsular polysaccharides using NaIO₄, and purification using a desalting column.

FIG. 7D illustrates the modification of GBS serotype II capsular polysaccharides with a hydrazine linker (ADH) and an active ester spacer (SIDEA).

### SEQUENCES

| SEQ ID NO | Description | Length |
|---|---|---|
| 1 | GBS Lumazine Synthase (LS) + N-terminal lysine + N-terminal His tag | 170aa |
| 2 | GBS ferritin KLL24083- unmodified | 155aa |
| 3 | Modified + N-terminal addition GBS ferritin KLL24083 (Cys to Ser at #4 and #124 [of SEQ ID NO:2] + N-terminal double lysine + N-term His tag see also SEQ ID NO: 24 | 169aa |
| 4 | subunit of S. pyogenes Dpr (RCSB PDB sequence) | 175 aa |
| 5 | subunit of S. pyogenes Dpr (NCBI reference NP_269604.1) | 175aa |
| 6 | GBS Ferritin DK-PW-092 - unmodified | 155aa |
| 7 | modified GBS Ferritin 092 subunit (Cys to Ser at #124) | 155aa |
| 8 | GBS Ferritin 14747- unmodified | 153aa |
| 9 | GBS 092 (Cys to Ser at #124) + C-terminal His Tag | 165aa |
| 10 | GBS Ferritin 14747 + C-terminal His Tag | 163aa |
| 11 | GBS Ferritin 14747 + N-terminal Helix | 175aa |
| 12 | GBS Ferritin 14747 + N-terminal Lysine-rich trimer & PADRE epitope | 184aa |
| 13 | GBS Ferritin LMG 14747 + N-term helix + C-term His Tag | 185aa |
| 14 | GBS Ferritin LMG 14747 + N-term Lysine-rich trimer + C-term His Tag | 194aa |
| 15 | N-terminal helix from S. pyogenes (Group A strep) | 25aa |
| 16 | Lysine rich helix | 21aa |
| 17 | PADRE epitope | 13aa |
| 18 | SEQ ID NO:7 (modified GBS 092) with first three aa replaced with S. pyogenes helix (SEQ ID NO: 15) | 177aa |
| 19 | His Tag w/flexible linker | 10aa |
| 20 | FLAG tag (Asp-Tyr-Lys-Asp-Asp-Asp-Asp-Lys) DYKDDDDK | 8aa |
| 21 | Strep tag (Ala-Trp-Arg-His-Pro-Gln-Phe-Gly-Gly) AWRHPQFGG | 9aa |
| 22 | Trp-Ser-His-Pro-Gln-Phe-Glu-Lys WSHPQFEK | 8aa |
| 23 | Trp-Ser-His-Pro-Gln-Phe-Glu-Lys-Gly-Gly-Gly-Ser-Gly-Gly-Gly-Ser-Gly-Gly-Gly-Ser- Trp-Ser-His-Pro-Gln-Phe-Glu-Lys WSHPQFEKGGGSGGGSGGGSWSHPQFEK | 28aa |
| 24 | Modified GBS Ferritin GenBank KLL24083 (Cys to Ser at #4 and #124 [of SEQ ID NO:2] | 155 aa |

GBS Sequences (‘modified’ refers to sequence of subunit per se, not to tags or N-terminal additions)

| | |
|---|---|
| Unmodified GBS | SEQ ID NO: 2 (KLL24083) |
| | SEQ ID NO: 6 (DK-PW-092) |
| | SEQ ID NO: 8 (14747) |
| Internal Modified | SEQ ID NO:7 (modified 092 subunit (Cys to Ser at #124)) |
| | SEQ ID NO:24 (modified KLL24083 subunit (Cys to Ser at #4 and #124)) |
| Tagged only | SEQ ID NO: 10 (14747 + C-terminal His Tag) |
| Tagged + Modified | SEQ ID NO: 9 (GBS modified 092 (Cys to Ser at #124) + C-terminal His Tag |
| N-term addition only | SEQ ID NO: 11 (GBS 14747 + N-terminal Helix) SEQ ID NO: 12 (GBS 14747 + N-terminal Lysine & PADRE epitope) |
| N-term + Modified | SEQ ID NO: 3 (GBS KLL24083 (Cys to Ser at #4 and #124 [of SEQ ID NO:2] + N-term double lysine + N-term His tag |
| | SEQ ID NO: 18 (modified GBS 092, + first three aa replaced with S. pyogenes helix |
| N-term + Tagged | SEQ ID NO: 13 (GBS 14747 + N-term helix + C-term His Tag) |
| | SEQ ID NO: 14 (GBS 14747 + N-term Lysine-rich trimer + C-term His Tag) |

### DETAILED DESCRIPTION

### Introduction

The present invention relates to: polypeptides capable of self-assembling into nanoparticles; nanoparticles (NP) comprising or consisting of such subunit polypeptides; and such NPs further comprising molecules attached to the exterior surface of the NP or contained within the hollow core of the NP.

The subunit polypeptides of the present invention are capable of self-assembly into roughly spherical nanoparticles, i.e., particles of less than about 100 nm in maximum diameter. Self-assembly is by non-covalent polypeptide-polypeptide interactions. In one embodiment, the NPs of the present invention are made of 12 polypeptide subunits.

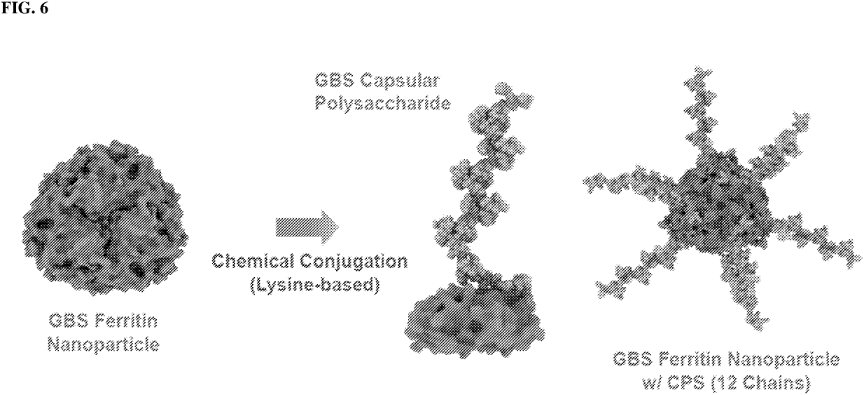

The polypeptides and NPs of the present invention may be used for any suitable purpose, such as for drug delivery by encapsulating drug molecules within the core of the NP. A further use is as a scaffold or carrier for displaying molecules of interest at the NP exterior surface. In one embodiment, the NP provides a scaffold for the display, at the exterior surface, of (a) antigenic non-polypeptide molecules (e.g., glycans or saccharides), (b) antigenic polypeptides, or (c) antigenic glycoconjugate molecules. Multiple copies of structurally defined antigenic epitopes can be displayed on the exterior surface of the NPs of the present invention; FIG. 6 depicts an exemplary embodiment of a nanoparticle of the present invention, where saccharide antigens are chemically conjugated to the surface of an NP.

### Ferritin NPs

Bacterial lumazine synthase (LS) has been investigated for use as an antigen-carrying protein particle. LS acts in the biosynthesis of riboflavin and is present in organisms including bacteria, plants, and eubacteria. Jardine et al. reported LS from the bacterium Aquifex aeolicus fused to an HIV gp120 antigen self-assembled into a 60-mer nanoparticle. Jardine et al., Science 340:711-716 (2013). Expression of wild-type A. aeolicus LS has been reported in E. coli; Jardine et al. described use of mammalian cells to produce LS nanoparticles comprising the HIV gp120 antigen.

H. pylori bacterial ferritin consists of 24 identical polypeptide subunits that self-assemble into a spherical nanoparticle. Li et al. reported preparation of a nucleotide sequence encoding a fusion of bacterial (H. pylori) ferritin subunit polypeptide, a rotavirus VP6 antigen, and a histidine tag to aid in purification, with expression in a prokaryotic (E. coli) system and removal of the His-tag. The expressed fusion polypeptides are described as self-assembling into spherical NPs displaying the rotavirus capsid protein VP6, and capable of inducing an immune response in mice. (Li et al., J Nanobiotechnol 17:13 (2019)). Wang et al. designed chimeric polypeptides comprising H. pylori ferritin and antigenic peptides from N. gonorrhoeae; the chimeric polypeptide is described as assembling into a 24-mer nanoparticle displaying the antigenic peptides on the NP exterior surface. (Wang et al., FEBS Open Bio 7(8):1196 (2017)). Kanekiyo et al. described a self-assembling recombinant bacterial (H. pylori) ferritin nanoparticle (24-mer), comprising fusions of the ferritin subunit polypeptide and influenza HA antigenic peptides, which displayed influenza HA trimers on its surface. (Kanekiyo et al., Nature 499(7456):102 (2013)).

Nanoparticles based on insect ferritin and comprising both heavy and light chain subunit polypeptides have been described for use in displaying, on the NP surface, trimeric antigens (WO2018/005558 (PCT/US2017/039595), Kwong et al.).

Li et al. described a nanoparticle made of recombinant fusion polypeptides comprising a human ferritin light-chain subunit and a short HIV-1 antigenic peptide attached to the amino terminus of the ferritin light-chain sequence, with self-assembly of these fusion polypeptides resulting in placement of the HIV-1 antigenic peptide at the exterior surface of the NP. Li et al., Ind. Biotechnol. 2:143-47 (2006)).

For vaccine formulations and to facilitate reaction with antigens which are not soluble themselves at high concentration, it is desirable for nanoparticles intended for use in vaccine compositions to have high solubility. Additionally, efficient manufacture is necessary for commercial uses; recombinantly expressed polypeptides and NPs should be capable of expression in high volume.

### Investigation of GBS Proteins (LS and Ferritin); Modification of GBS Ferritin

The present inventors investigated NPs based on recombinantly produced Streptococcus agalactiae proteins (lumazine synthase and ferritin). Streptococcus agalactiae is also known as Group B Streptococcus (GBS). The present inventors modified the polypeptide subunit of GBS lumazine synthase by adding at the N-terminal a His-Tag and two lysine residues (SEQ ID NO:1); expression was attempted in both a bacterial and a mammalian expression system (See Example 1). The present inventors additionally modified a naturally-occurring GBS ferritin subunit sequence by substituting a serine for each of two cysteine residues and adding a His-Tag and two lysine residues at the N-terminus (SEQ ID NO:3); expression was attempted in a bacterial expression system (See Example 2).

Using in silico database searches, the present inventors additionally identified naturally-occurring GBS proteins with homology to the Streptococcus pyogenes DPS-like peroxide resistance polypeptide subunit (SEQ ID NO: 8 (14747) and SEQ ID NO:6 (092)). The GBS proteins were modified to provide SEQ ID NOs: 7, 9, 10, 11, 12, 13, 14, and 18.

### Polypeptides and NPs

One embodiment of the present invention is polypeptides capable of self-assembly into an NP (referred to as “subunit” polypeptides herein), and variants of such polypeptides. The polypeptide subunit may comprise or consist of a naturally-occurring GBS polypeptide from any strain, or modifications or variants of such polypeptides. For example, the subunit polypeptide may consist of or comprise a naturally-occurring GBS subunit polypeptide to which a purification tag, an N-terminus alpha helix (such as the sequence from S. pyogenes Dpr, SEQ ID NO:15), or a N-terminus lysine-rich trimeric helix (such as SEQ ID NO:16) has been added. Alternatively, the sequence of the GBS subunit per se may be modified in comparison to a naturally-occurring GBS sequence, e.g., by amino acid deletions, insertions, or substitutions; to such modified subunit sequences may further be added one or more N-terminal or C-terminal sequences, such as a purification tag, an N-terminus helix (such SEQ ID NO:15), or a N-terminus lysine-rich trimeric helix (such as SEQ ID NO:16).

In one aspect, a polypeptide of the present invention comprises a sequence from a naturally occurring GBS ferritin (or ferritin-like) subunit polypeptide, or modifications or variants thereof. Modifications may be for the purpose of reducing aggregation or imparting other characteristics desirable for production. Modifications may include deletions of one or more amino acids, insertions of one or more amino acids, or substitutions of one or more amino acids. For example, a subunit having SEQ ID NO:7 is modified compared to the ferritin subunit of strain DK-PW-092 (sequence provided at GenBank KLL27267.1; SEQ ID NO: 6)). The modified sequence contains a Cysteine to Serine amino acid substitution at position #124.

In one aspect, polypeptides of the present invention comprise one or more Cysteine to Serine amino acid substitution(s), compared to a naturally-occurring GBS sequence. Polypeptides of the invention may additionally or alternatively contain one or more N-terminal or C-terminal sequences, including but not limited to poly-histidine tags, short (less than 20, less than ten, or less than five amino acids in length) linker sequences, and polypeptide sequences that form alpha helical structures (e.g., SEQ ID NO:15). Such modifications may replace a single amino acid, or a short (ten or fewer amino acids, five or fewer amino acids, or three or two amino acids) contiguous sequence of amino acids of the subunit sequence or modified subunit sequence (see, e.g., SEQ ID NOs: 8 and 11, where SEQ ID NO: 11 contains an N-terminal helix substituted for the first three N-terminal amino acids of SEQ ID NO: 8.

When referring to amino acid positions (residues) in a GBS subunit polypeptide sequence, numbering herein is given in relation to the amino acid sequence of SEQ ID NO:7 unless otherwise specified. Therefore, when referring to “comparable” or “corresponding” amino acid positions or specific amino acids in subunit polypeptides (also relevant in the context of nucleic acids), residues “comparable” or “corresponding” to those in a reference sequence can be determined by one of ordinary skill in the art using known information, and by sequence alignment using readily available and well-known alignment algorithms (such as BLAST, using default settings; ClustalW2, using default settings; or algorithm disclosed by Corpet, Nucleic Acids Research, 1998, 16(22):10881-10890, using default parameters). Accordingly, when referring to a “GBS subunit polypeptide”, it is to be understood as a GBS subunit polypeptide from any GBS strain unless otherwise denoted. The actual residue location number and residue identity may have to be adjusted for polypeptides from GBS other than DK-PW-092 strain. Orientation within a polypeptide is generally recited in an N-terminal to C-terminal direction, defined by the orientation of the amino and carboxy moieties of individual amino acids. Polypeptides are translated from the N-terminal or amino-terminus towards the C-terminal or carboxy-terminus.

Amino acid substitutions may be conservative substitutions. Amino acids are commonly classified into distinct groups according to their side chains. For example, some side chains are considered non-polar, i.e. hydrophobic, while some others are considered polar, i.e. hydrophilic. Alanine (A), glycine (G), valine (V), leucine (L), isoleucine (I), methionine (M), proline (P), phenylalanine (F) and tryptophan (W) are considered to be hydrophobic amino acids, while serine (S), threonine (T), asparagine (N), glutamine (Q), tyrosine (Y), cysteine (C), lysine (K), arginine (R), histidine (H), aspartic acid (D) and glutamic acid (E) are considered to be polar amino acids. Regardless of their hydrophobicity, amino acids are also classified into subgroups based on common properties shared by their side chains. For example, phenylalanine, tryptophan and tyrosine are jointly classified as aromatic amino acids and will be considered as aromatic amino acids within the meaning of the present invention. Aspartate (D) and glutamate (E) are among the acidic or negatively charged amino acids, while lysine (K), arginine (R) and histidine (H) are among the basic or positively charged amino acids, and they will be considered as such in the sense of the present invention. Hydrophobicity scales are available which utilize the hydrophobic and hydrophilic properties of each of the 20 amino acids and allocate a hydrophobic score to each amino acid, creating thus a hydrophobicity ranking. As an illustrative example only, the Kyte and Dolittle scale may be used (Kyte et al. 1982. J. Mol. Bio. 157: 105-132). This scale allows one skilled in the art to calculate the average hydrophobicity within a segment of predetermined length.

Polypeptide alpha helices are known in the art; they are secondary polypeptide structures where backbone N—H groups bond to backbone C═O groups to form a right-hand helix. Helices may be from about ten amino acids to about 100 amino acids in length, from about ten amino acids to about 80 amino acids in length, from about ten amino acids to about 60 amino acids in length, from about ten amino acids to about 50 amino acids in length, from about ten amino acids to about 40 amino acids in length, from about ten amino acids to about 30 amino acids in length, from about 15 amino acids to about 100 amino acids in length, from about 15 amino acids to about 80 amino acids in length, from about 15 amino acids to about 60 amino acids in length, from about 15 amino acids to about 50 amino acids in length, from about 15 amino acids to about 40 amino acids in length, from about 15 amino acids to about 30 amino acids in length, from about 20 amino acids to about 100 amino acids in length, from about 20 amino acids to about 80 amino acids in length, from about 20 amino acids to about 60 amino acids in length, from about 20 amino acids to about 50 amino acids in length, from about 20 amino acids to about 40 amino acids in length, or from about 20 amino acids to about 30 amino acids in length.

The polypeptides of the present invention may contain an amino acid sequence known as a “tag”, which facilitates purification (e.g. a polyhistidine-tag to allow purification on a nickel-chelating resin). Examples of affinity-purification tags include, e.g., 6xHis tag (hexahistidine (SEQ ID NO: 25), binds to metal ion), maltose-binding protein (MBP) (binds to amylose), glutathione-S-transferase (GST) (binds to glutathione), FLAG tag (Asp-Tyr-Lys-Asp-Asp-Asp-Asp-Lys (SEQ ID NO: 20), binds to an anti-flag antibody), Strep tag (Ala-Trp-Arg-His-Pro-Gln-Phe-Gly-Gly (SEQ ID NO: 21), or Trp-Ser-His-Pro-Gln-Phe-Glu-Lys (SEQ ID NO: 22), or Trp-Ser-His-Pro-Gln-Phe-Glu-Lys-Gly-Gly-Gly-Ser-Gly-Gly-Gly-Ser-Gly-Gly-Gly-Ser- Trp-Ser-His-Pro-Gln-Phe-Glu-Lys (SEQ ID NO:23) binds to streptavidin or a derivative. The tag may be removed (enzymatically or through other means) prior to NP assembly, or may be retained on the subunit and thus contained in the NP.

A “variant” of a reference polypeptide sequence includes amino acid sequences having one or more amino acid substitutions, insertions and/or deletions when compared to the reference sequence. The variant may comprise an amino acid sequence which is at least 70%, at least 75%, at least 80%, at least 85%, at least 90%, at least 95%, at least 96%, at least 97%, at least 98%, or at least 99% identical to a full-length reference polypeptide.

While their structure is distinct from non-modified subunit polypeptides, the modified polypeptides of the present invention maintain the ability to self-assemble into a nanoparticle protein, such as a nanoparticle made up of twelve copies of the same modified subunit polypeptide. Self-assembly of NPs refers to the oligomerization of polypeptide subunits into an ordered arrangement, driven by non-covalent interactions. Such noncovalent interactions may be any of electrostatic interactions, Π-interactions, van der Waals forces, hydrogen bonding, hydrophobic effects, or any combination thereof.

The polypeptides of the invention may be modified to introduce amino acid residues known in the art as capable of being chemically conjugated to a heterologous molecule, such as an antigenic polypeptide, polysaccharide or glycoconjugate. A further embodiment of the present invention is NPs consisting of or comprising such polypeptides, and such NPs comprising heterologous molecules chemically conjugated to the NP.

In another embodiment, polypeptides of the invention comprise an N-terminal capture sequence. The capture sequence may be located directly at the N-terminus of the subunit sequence or attached by a short amino acid linker sequence. In another embodiment, one, two, three or more (up to ten) N-terminal amino acid residues of the subunit polypeptide sequence is replaced by the capture sequence. The resulting assembled NP will comprise capture sequences (“tag”) on the surface of the NP, which can then pair with a corresponding “dock” sequence on the molecule which is desired to be displayed on the surface of the NP. A suitable capture sequence is one that will specifically bind, either covalently or non-covalently, to the desired target molecule. Suitable polypeptide linkers include linkers of two or more amino acids. An illustrative polypeptide linker is one or more multimers of GGS or GSS.

The present invention further provides nanoparticles consisting of or comprising polypeptides of the invention. In one aspect, the nanoparticle consists of subunits with the same amino acid sequence (homomultimeric). Alternatively, the nanoparticle may consist of, or comprise, polypeptide subunits that differ in amino acid sequence (heteromultimeric). The NPs may display one or more heterologous molecule(s) on the exterior NP surface; the displayed molecule may be antigenic. A single NP may display multiple copies of the same antigen, or may display different antigens.

### Polynucleotide Molecules

A further aspect of the present invention is polynucleotide molecules encoding the polypeptides of the present invention. The nucleic acid molecule may comprise RNA or DNA. Such recombinant nucleic acid sequences may comprise additional sequences useful for promoting expression or purification of the encoded polypeptide.

Polynucleotide molecules encoding the polypeptides of the invention may be codon optimized for expression in a selected prokaryotic or eukaryotic host cell. By “codon optimized” is intended modification with respect to codon usage that may increase translation efficacy and/or half-life of the nucleic acid.

### Sequence Identity

According to the present invention, two proteins having a high degree of identity have amino acid sequences at least 80% identical, at least 85% identical, at least 87% identical, at least 90% identical, at least 92% identical, at least 94% identical, at least 96% identical, at least 98% identical or at least 99% identical. It will be understood by those of skill in the art that the similarity between two polypeptide sequences (or polynucleotide sequences), can be expressed in terms of the similarity between the sequences, otherwise referred to as sequence identity. Sequence identity is frequently measured in terms of percentage identity (or similarity); the higher the percentage, the more similar are the primary structures of the two sequences. In general, the more similar the primary structures of two polypeptide (or polynucleotide) sequences, the more similar are the higher order structures resulting from folding and assembly. Methods of determining sequence identity are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations. The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Sequence identity between polypeptide sequences is preferably determined by pairwise alignment algorithm using the Needleman-Wunsch global alignment algorithm (Needleman and Wunsch, A General Method Applicable to the Search for Similarities in the Amino Acid Sequence of Two Proteins, 1970 J. Mol. Biol. 48(3): 443-453), using default parameters (e.g. with Gap opening penalty = 10.0, and with Gap extension penalty = 0.5, using the EBLOSUM62 scoring matrix). This algorithm is conveniently implemented in the needle tool in the EMBOSS package (Rice et al., EMBOSS: The European Molecular Biology Open Software Suite, 2000 Trends Genetics 16: 276-277). Sequence identity should be calculated over the entire length of the polypeptide sequence of the invention.

### Expression Methods

The polypeptides of the present invention may be produced any suitable means, including by recombinant expression production or by chemical synthesis. Polypeptides of the invention may be recombinantly expressed and purified using any suitable method as is known in the art, and the product analyzed using methods as known in the art, e.g., by crystallography, Dynamic Light Scattering (DLS), Nano-Differential Scanning Fluorimetry (Nano-DSF), and Electron Microscopy, to confirm modified sequences assemble into nanoparticles.

Thus a further embodiment of the invention is a method of producing a polypeptide (or NP) according to the invention. The method comprises the steps of (a) culturing a recombinant host cell according to this aspect of the invention under conditions conducive to the expression of the polypeptide. The method may further comprise recovering, isolating, or purifying the expressed polypeptide. In one embodiment, multiple copies of a subunit polypeptide of the present invention are expressed in a host cell, where the subunit polypeptides self-assemble into a multimeric nanoparticle within the host cell. The assembled NP can then be recovered, isolated or purified from the cell or the culture medium in which the cell is grown.

Methods of recombinant expression suitable for the production of the polypeptides of the present invention are known in the art. The expressed polypeptide may include a purification tag, a linker, a capture sequence (one component of a tag-capture system), or a “display” polypeptide (as described herein). Various expression systems are known in the art, including those using human (e.g., HeLa) host cells, mammalian (e.g., Chinese Hamster Ovary (CHO)) host cells, prokaryotic host cells (e.g., E. coli), or insect host cells. The host cell is typically transformed with the recombinant nucleic acid sequence encoding the desired polypeptide product, cultured under conditions suitable for expression of the product, and the product purified from the cell or culture medium. Cell culture conditions are particular to the cell type and expression vector, as is known in the art.

When a recombinant host cell of the present invention is cultured under suitable conditions, the recombinant nucleic acid expresses a NP subunit polypeptide as described herein. The polypeptide self-assembles within the cell to provide a nanoparticle. Suitable host cells include, for example, insect cells (e.g., Aedes aegypti, Autographa californica, Bombyx mori, Drosophila melanogaster, Spodoptera frugiperda, and Trichoplusia ni), mammalian cells (e.g., human, non-human primate, horse, cow, sheep, dog, cat, and rodent (e.g., hamster)), avian cells (e.g., chicken, duck, and geese), bacteria (e.g., E. coli, Bacillus subtilis, and Streptococcus spp.), yeast cells (e.g., Saccharomyces cerevisiae, Candida albicans, Candida maltosa, Hansenual polymorpha, Kluyveromyces fragilis, Kluyveromyces lactis, Pichia guillerimondii, Pichia pastoris, Schizosaccharomyces pombe and Yarrowia lipolytica), Tetrahymena cells (e.g., Tetrahymena thermophila) or combinations thereof.

Host cells can be cultured in conventional nutrient media modified as appropriate and as will be apparent to those skilled in the art (e.g., for activating promoters). Culture conditions, such as temperature, pH and the like, may be determined using knowledge in the art, see e.g., Freshney (1994) Culture of Animal Cells, a Manual of Basic Technique, third edition, Wiley- Liss, New York and the references cited therein. In bacterial host cell systems, a number of expression vectors are available including, but not limited to, multifunctional E. coli cloning and expression vectors such as BLUESCRIPT (Stratagene) or pET vectors (Novagen, Madison WI). In mammalian host cell systems, a number of expression systems, including both plasmids and viral-based systems, are available commercially.

Eukaryotic or microbial host cells expressing polypeptides of the invention can be disrupted by any convenient method (including freeze-thaw cycling, sonication, mechanical disruption), and polypeptides and/or NPs can be recovered and purified from recombinant cell culture by any suitable method known in the art (including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxyapatite chromatography, and lectin chromatography). High performance liquid chromatography (HPLC) can be employed in the final purification steps.

In general, and using methods as are known in the art, expression of a recombinantly encoded polypeptide of the present invention involves preparation of an expression vector comprising a recombinant polynucleotide under the control of one or more promoters, such that the promoter stimulates transcription of the polynucleotide and promotes expression of the encoded polypeptide. “Recombinant Expression” as used herein refers to such a method.

In a further aspect, the present invention provides recombinant expression vectors comprising a recombinant nucleic acid sequence of any embodiment of the invention operatively linked to a suitable control sequence. “Recombinant expression vector” includes vectors that operatively link a nucleic acid coding region or gene to any control sequences capable of effecting expression of the gene product. “Control sequences” are nucleic acid sequences capable of effecting the expression of the nucleic acid molecules and need not be contiguous with the nucleic acid sequences, so long as they function to direct the expression thereof. Recombinant expression vectors can be of any type known in the art, including but not limited to plasmid and viral-based expression vectors. The control sequence used to drive expression of the disclosed nucleic acid sequences in a mammalian system may be constitutive or inducible. The construction of expression vectors for use in transfecting prokaryotic cells is also well known in the art. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, Tex.). The expression vector must be replicable in the selected host organism either as an episome or by integration into host chromosomal DNA. In non-limiting embodiments, the expression vector is a plasmid vector or a viral vector. Expression vectors suitable for use in a given host-expression system and containing the encoding nucleic acid sequence and transcriptional/translational control sequences, may be made by any suitable technique as is known in the art. Typical expression vectors contain suitable promoters, enhancers, and terminators that are useful for regulation of the expression of the coding sequence(s) in the expression construct. The vectors may also comprise selection markers to provide a phenotypic trait for selection of transformed host cells (such as conferring resistance to antibiotics such as ampicillin or neomycin). Nucleic acid or vector modification may be undertaken in a manner known by the art, see e.g., WO 2012/049317 (corresponding to US 2013/0216613) and WO 2016/092460 (corresponding to US 2018/0265551). For example, the nucleic acid sequence encoding an NP subunit polypeptide as described herein is cloned into a vector suitable for introduction into the selected cell system, e.g., bacterial or mammalian cells (e.g., CHO cells). Transformed cells are expanded, e.g., by culturing.

In a further embodiment, the present invention provides recombinant host cells that comprise a recombinant expression vector of the present invention. Suitable host cells can be either prokaryotic or eukaryotic, such as mammalian cells. The cells can be transiently or stably transfected. Such transfection of expression vectors into prokaryotic and eukaryotic cells can be accomplished via any technique known in the art, including but not limited to standard bacterial transformations, calcium phosphateco-precipitation, electroporation, or liposome mediated-, DEAE dextran mediated-, polycationic mediated-, or viral mediated transfection or transduction. (See, for example, Molecular Cloning: A Laboratory Manual (Sambrook, et al., 1989, Cold Spring Harbor Laboratory Press; Culture of Animal Cells: A Manual of Basic Technique, 2.sup.nd Ed. (R. I. Freshney.1987. Liss, Inc. New York, N.Y.).

The expressed subunit polypeptides assemble into nanoparticles, and the NPs are recovered (e.g., purified, isolated, or enriched).

### Purification

The term “purified” as used herein refers to the separation or isolation of a defined product (e.g., a recombinantly expressed polypeptide) from a composition containing other components (e.g., a host cell or host cell medium). A polypeptide composition that has been fractionated to remove undesired components, and which composition retains its biological activity, is considered purified. A purified polypeptide retains its biological activity. Purified is a relative term and does not require that the desired product be separated from all traces of other components. Stated another way, “purification” or “purifying” refers to the process of removing undesired components from a composition or host cell or culture. Various methods for use in purifying polypeptides and NPs of the present invention are known in the art, e.g., centrifugation, dialysis, chromatography, gel electrophoresis, affinity purification, filtration, precipitation, antibody capture, and combinations thereof. The polypeptides NPs of the present invention may be expressed with a tag operable for affinity purification, such as a 6xHistidine tag (SEQ ID NO: 25) as is known in the art. A His-tagged polypeptide may be purified using, for example, Ni-NTA column chromatography or using anti-6xHis antibody (SEQ ID NO: 25) fused to a solid support.

Thus, the term “purified” does not require absolute purity; rather, it is intended as a relative term. A “substantially pure” preparation of polypeptides (or nanoparticles) or nucleic acid molecules is one in which the desired component represents at least 50% of the total polypeptide (or nucleic acid) content of the preparation. In certain embodiments, a substantially pure preparation will contain at least 60%, at least 70%, at least 80%, at least 85%, at least 90%, at least 95%, at least 97%, at least 98%, or at least 99% or more of the total polypeptide (or nucleic acid) content of the preparation. Methods for quantifying the degree of purification of expressed polypeptides are known in the art and include, for example, determining the specific activity of an active fraction, or assessing the number of polypeptides within a fraction by SDS/PAGE analysis.

Thus, in the sense of the present invention, a “purified” or an “isolated” biological component (such as a polypeptide, an NP, or a nucleic acid molecule) has been substantially separated or purified away from other biological components in which the component naturally occurs or was recombinantly produced. The term embraces polypeptides, NPs, and nucleic acid molecules prepared by chemical synthesis as well as by recombinant expression in a host cell.

### Antigenic Display

Molecules, including antigenic molecules, attached to the exterior surface of an NP of the present invention may be referred to herein as “display” or “displayed” molecules. Antigen-displaying nanoparticles preferably display multiple copies of antigenic molecules in an ordered array. It is theorized that an ordered multiplicity of antigens presented on a NP allows multiple binding events to occur simultaneously between the NP and a host’s cells, which favors the induction of a potent host immune response. See e.g., Lopez-Sagaseta et al., Comput Struct Biotechnol J, 14:58-68 (2016).

Fusions of subunit + antigen: One embodiment of the present invention is fusion proteins comprising an NP polypeptide subunit sequence with a heterologous antigenic polypeptide attached N-terminally to the subunit sequence. The antigenic polypeptide may be covalently attached directly to the N-terminal amino acid of the subunit sequence, or a short (less than about 20, less than about 15, less than about 10, or less than about 5 amino acids) peptide linker sequence may be placed between the antigenic polypeptide and the subunit sequences. A further embodiment of the present invention is NPs comprising or consisting of such fusion proteins, and such fusion proteins are themselves referred to as subunit polypeptides herein. Self-assembly of NP subunits places their N-termini at the outer surface, and C-termini at the inner surface, of the assembled NP. Antigenic polypeptides attached to a subunit N-terminus are thus displayed at the exterior surface of the assembled NP.

A further embodiment of the present invention provides recombinant polynucleotide sequences encoding such fusion proteins.

### Methods of Conjugation

Displayed molecules may be incorporated onto or attached to the NPs of the present invention by any suitable means.

Genetic construct: In one embodiment, a nucleotide construct is prepared that recombinantly expresses a contiguous polypeptide sequence comprising both the NP subunit sequence and the polypeptide sequence of the displayed molecule, i.e., expresses a fusion polypeptide comprising both the NP subunit polypeptide and a display polypeptide. The encoded fusion polypeptides self-assemble into the NP. By positioning the display molecule at the N-terminus of the fusion polypeptide, the display molecule is located on the exterior surface of the assembled NP.

Chemical conjugation: Functional groups present on the NP subunit polypeptides can be used for site-specific conjugation of display molecules. Amino acid side-chain groups used for conjugation include amino group on lysine, thiol on cysteine, carboxylic acid on aspartic acids and glutamic acids, and hydroxyl moiety on tyrosine. Heterobifunctional crosslinkers are available for protein conjugation. Primary amines on proteins can be conjugated to carboxylic acids on another protein using 1-ethyl-3-(-3-dimethylaminopropyl) carbodiimide (EDC) crosslinkers, typically in combination with N-hydroxysuccinimide (NHS). The side-chain amino groups of lysine residues are nucleophiles, so lysine residues exposed at the NP surface have large solvent accessibility and can be used as sites for conjugation to display molecules.

One or more selected amino acid residues within a subunit polypeptide sequence may be modified using methods known in the art to provide a site suitable for chemical conjugation at the NP exterior surface, where such modification does not disrupt the polypeptide activity.

One embodiment of the present invention is a multimeric polypeptide nanoparticle consisting of or comprising the polypeptides of the invention, the NP exhibiting a plurality of surface sites suitable for chemical conjugation of a display molecule, such as a polypeptide or saccharide antigen.

One embodiment of the present invention is an NP consisting of or comprising polypeptides of the invention, where one or more display molecules are chemically conjugated to lysine residues present at the exterior surface of the NP. The display molecule(s) may be a polypeptide, glycan, or glycoconjugate, or combinations thereof.

Tag - dock: One embodiment of the present invention is an NP comprising or consisting of subunit polypeptides of the invention, and displaying heterologous molecule(s) on the exterior surface of the NP, where the displayed molecule(s) is conjugated to the NP subunit using a split-protein binding system (tag-capture system), such as a SpyCatcher-SpyTag binding system (SPYBiotech, Oxford, England). Accordingly, one aspect of the present invention is a fusion polypeptide comprising or consisting of an NP subunit sequence and one sequence from a split-protein binding system; a further aspect of the invention is NPs comprising or consisting of such fusion polypeptides, where the split-protein binding system sequence is on the exterior surface of the NP. When such an NP is exposed to a heterologous molecule containing the other sequence of the binding pair, an isopeptide bond forms and the heterologous molecule becomes covalently attached to the surface of the NP. See, e.g., Brune et al., Frontiers in Immunology 9:1432 (2018); Reddington et al., Curr Opinion Chem Biol 29:94-99 (2015); Brune et al., Bioconjugate Chem 28(5):1544-51 (2017); Tan et al., PLOS One 11(10):e0165074 (2016).

### Antigens

One embodiment of the present invention is nanoparticles comprising or consisting of polypeptides of the invention, where the nanoparticles display one or more heterologous (as compared to the polypeptide subunit proteins) molecules on the exterior surface of the nanoparticle. Where said displayed molecules are polypeptides, they may be expressed as part of the polypeptide (i.e., as a fusion protein), such that self-assembly of the NP results in display on the NP exterior surface. Alternatively, a polypeptide display molecule may be attached to the assembled NP, for example, by chemical conjugation as discussed herein and as known in the art.

In a further embodiment of the present invention the display molecule is a poly- or oligo-saccharide, such as a bacterial capsular polysaccharide; the saccharide may be linked to a protein carrier to provide a glycoconjugate. Capsular polysaccharides are important immunogens found on the surface of bacteria involved in various bacterial diseases. They have proved useful in eliciting immune responses in mammalian subjects especially when linked to carrier proteins (glycoconjugates). In some embodiments of the present invention, the displayed antigen is an oligo/polysaccharide derived from a bacterial pathogen and in particular may be derived from bacterial capsular saccharide or lipooligosaccharide (LOS) or lipopolysaccharide (LPS). For example, the oligo/polysaccharide may be derived from a bacterial pathogen selected from the group consisting of: S. agalactiae, Haemophilus influenzae type b (“Hib”); Neisseria meningitidis (including serotypes A, C, W and/or Y); Streptococcus pneumoniae (including serotypes 1, 2, 3, 4, 5, 6A, 6B, 7F, 8, 9N, 9V, 10A, 11A, 12F, 14, 15B, 15C, 17F, 18C, 19A, 19F, 20, 22F, 23F and/or 33F); Staphylococcus aureus, Bordetella pertussis, and Salmonella typhi.

Further bacterial antigens for use in the present invention include those from by Escherichia species, Shigella species, Klebsiella species, Salmonella species, Yersinia species, Helicobacter species, Proteus species, Pseudomonas species, Corynebacterium species, Streptomyces species, Streptococcus species, Enterococcus species, Staphylococcus species, Bacillus species, Clostridium species, Listeria species, or Campylobacter species.

### Group B Streptococcus

Streptococcus agalactiae (also known as “Group B Streptococcus” or “GBS”) is a β-hemolytic, encapsulated Gram-positive microorganism that is a major cause of neonatal sepsis and meningitis, particularly in infants born to women carrying the bacteria (Heath & Schuchat (2007)). The GBS capsule is a virulence factor that assists the bacterium in evading human innate immune defences. The GBS capsule consists of high molecular weight polymers made of multiple identical repeating units of four to seven monosaccharides and including sialic acid (N-acetylneuraminic acid) residues. GBS can be classified into ten serotypes (Ia, Ib, II, III, IV, V, VI, VII, VIII, and IX) based on the chemical composition and the pattern of glycosidic linkages of the capsular polysaccharide repeating units. Non-typeable strains of GBS are also known to exist. Description of the structure of GBS CPS may be found in the published literature (see e.g., WO2012/035519). The capsular polysaccharides of different serotypes are chemically related, but are antigenically very different.

GBS capsular polysaccharides (also referred to as capsular saccharides) have been investigated for use in vaccines. However, saccharides are T-independent antigens and are generally poorly immunogenic. Covalent conjugation to a carrier molecule (such as a protein carrier) can convert T-independent antigens into T-dependent antigens, thereby enhancing memory responses and allowing protective immunity to develop. Glycoconjugate vaccines for each of GBS serotypes Ia, Ib, II, III, IV and V have separately been shown to be immunogenic in humans. Multivalent GBS vaccines are described, e.g., in WO2016-178123, WO2012-035519, and WO2014-053612. Clinical studies using monovalent or bivalent GBS glycoconjugate vaccine (GBS CPS conjugated to a protein carrier) have previously been conducted with both non-pregnant adults and pregnant women. See, e.g., Paoletti et al., Infect Immun., 64(2):677 (1996); Baker et al., J Infectious Dis, 179:142 (1999); Baker et al., J Infectious Dis, 182:1129 (2000); Baker et al., J Infectious Dis 188:66 (2003); Baker et al., J Infectious Dis, 189:1103 (2004).

In one embodiment of the present invention, the antigen displayed on the NP is a GBS capsular polysaccharide or immunogenic fragment thereof. The GBS capsular polysaccharide may be selected from any serotype, including Ia, Ib, II, III, IV and V. A single NP may display polysaccharides from more than one GBS serotype.

In one embodiment of the present invention, NPs display GBS protein antigens. Nearly all GBS strains express a protein which belongs to the so-called alpha-like proteins (Alps), of which Ca, Alp1, Alp2, Alp3, Rib, and Alp4 are known to occur in GBS. See e.g. Maeland et al., Clin Vaccine Immunol 22(2): 153-59 (2015). GBS protein antigens, including Alp3, and Rib proteins have been investigated as vaccine components (see e.g., Gravekamp et al., Infect Immun. 67:2491-6 (1999); Erdogan et al., Infect Immun. 70:803-11 (2002).) Additional GBS protein antigens include immunogenic fusion proteins comprising the N-terminal regions of two or more GBS proteins (such as Rib, AlpC, Alp1, Alp2, Alp3 or Alp4) (see e.g., WO2017/068112; WO2008/127179).

GBS pilus proteins are long filamentous structures protruding from the bacterial surface, which function in bacterial virulence and disease pathogenesis. Pili are composed of three structural proteins, the major pilus subunit (backbone protein, BP) that forms the pilus shaft and two ancillary proteins (AP1 and AP2). Nuccitelli et al. reported that BP-2a variants share four similar Ig-like domains (D1 to D4), and a D3 domain of BP-2a is a major epitope for a protective immune response. They further developed an immunogenic chimeric protein comprising six D3 domains from six BP-2a variants. Nuccitelli et al., Proc Natl Acad Sci U S A, 108(25):10278-83 (2011). See also WO 2011/121576; WO 2016/020413. GBS pilus proteins, and immunogenic fragments thereof, may be displayed by NPs of the present invention.

In one embodiment of the present invention, NPs display glycoconjugate antigens comprising a GBS protein and a GBS polysaccharide. See, e.g., WO2017/114655; WO 2017/068112;

### Compositions

A further embodiment of the present invention is immunogenic compositions or pharmaceutical compositions, such as vaccines. The pharmaceutical compositions comprise NPs of the present invention displaying antigens, and a pharmaceutically acceptable diluent, carrier or excipient. An “immunogenic composition” is a composition of matter suitable for administration to a human or non-human mammalian subject and which, upon administration of an immunologically effective amount, elicits a specific immune response, e.g., against an antigen displayed on the NP. As such, an immunogenic composition includes one or more antigens (for example, polypeptide antigens or saccharide antigens from Group B Streptococcus), or immunogenic fragments or antigenic epitopes thereof. An immunogenic composition can also include one or more additional components capable of enhancing an immune response, such as an excipient, carrier, and/or adjuvant.

In certain instances, immunogenic compositions are administered to elicit an immune response that protects the subject against infection by a pathogen, or decreases symptoms or conditions induced by a pathogen. In the context of this disclosure, the term immunogenic composition will be understood to encompass compositions that are intended for administration to a subject or population of subjects for the purpose of eliciting a protective or palliative immune response against GBS.

Numerous pharmaceutically acceptable diluents and carriers and/or pharmaceutically acceptable excipients are known in the art and are described, e.g., in Remington’s Pharmaceutical Sciences, by E. W. Martin, Mack Publishing Co., Easton, PA, 15th Edition (1975). The adjective “pharmaceutically acceptable” indicates that the diluent, or carrier, or excipient, is suitable for administration to a subject (e.g., a human or non-human mammalian subject). In general, the nature of the diluent, carrier and/or excipient will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. In certain formulations (for example, solid compositions, such as powder forms), a liquid diluent is not employed. In such formulations, non-toxic solid carriers can be used, including for example, pharmaceutical grades of trehalose, mannitol, lactose, starch or magnesium stearate. Suitable carriers are typically large, slowly metabolized macromolecules such as proteins (e.g., nanoparticles), polysaccharides, polylactic acids, polyglycolic acids, polymeric amino acids, amino acid copolymers, lipid aggregates (such as oil droplets or liposomes), and inactive virus particles. Such carriers are well known in the art.

Accordingly, suitable excipients and carriers can be selected by those of skill in the art to produce a formulation suitable for delivery to a subject by a selected route of administration.

The immunogenic compositions of the invention are conventionally administered parenterally, e.g., by injection, either subcutaneously, intraperitoneally, transdermally, or intramuscularly. Dosage treatment may be a single dose schedule or a multiple dose schedule. The immunogenic composition may be administered in conjunction with other immunoregulatory agents. Any suitable route of administration can be used, and administered according to any suitable schedule.

Immunogenic compositions of the present invention may additionally include one or more adjuvants. An “adjuvant” is an agent that enhances the production of an immune response in a non-specific manner. Common adjuvants include suspensions of minerals (alum, aluminum hydroxide, aluminum phosphate); saponins such as QS21; emulsions, including water-in-oil, and oil-in-water (and variants thereof, including double emulsions and reversible emulsions), liposaccharides, lipopolysaccharides, immunostimulatory nucleic acid molecules (such as CpG oligonucleotides), liposomes, Toll Receptor agonists (particularly, TLR2, TLR4, TLR⅞ and TLR9 agonists), and various combinations of such components.

Preparation of immunogenic compositions, such as vaccines, including those for administration to human subjects, is generally described in Pharmaceutical Biotechnology, Vol.61 Vaccine Design-the subunit and adjuvant approach, edited by Powell and Newman, Plenum Press, 1995. New Trends and Developments in Vaccines, edited by Voller et al., University Park Press, Baltimore, Maryland, U.S.A. 1978.

### Prophylactic and Therapeutic Uses

A further aspect of the present invention is a method of inducing an immune response in a subject, where said immune response is specific for a molecule displayed on the surface of NPs of the present invention. The method comprises administering to a subject an effective amount of the NPs displaying the molecule to which an immune response is desired. In one embodiment the display molecule is a bacterial antigen; the subject may have a bacterial infection at the time of administration, or the administration may be given prophylactically to a subject who does not have a bacterial infection at the time of administration.

A further aspect of the present invention is a method of treating and/or preventing a bacterial infection of a subject comprising administering to the subject an NP as described herein that displays antigens from the infecting organisms, where said antigens can induce a protective or therapeutic immune response. Such NPs may be within an immunogenic or pharmaceutical composition as described herein. In a specific embodiment, the NPs and compositions described herein are used in the prevention of infection of a subject (e.g., human subjects) by Group B Streptococcus (S. agalactiae).

Thus, in one embodiment, the NPs and compositions of the present invention are utilized in methods of immunizing a subject to achieve a protective (prophylactic) immune response, or as a therapeutic measure (i.e., directed against an existing disease in the subject).

### Embodiments

The various features which are referred to in individual sections above apply, as appropriate, to other sections. Consequently, features specified in one section may be combined with features specified in other sections, as appropriate. Those skilled in the art will recognize or be able to ascertain using no more than routine experimentation, many equivalents to the specific embodiments of the invention (or aspects of the disclosure) described herein. Such equivalents are intended to be encompassed by the following:

C1. A polypeptide capable of self-assembling into a nanoparticle, said polypeptide comprising or consisting of a sequence having at least 80% sequence identity, at least 85% sequence identity, at least 87% sequence identity, at least 90% sequence identity, at least 92% sequence identity, at least 94% sequence identity, at least 95% sequence identity, at least 96% sequence identity, at least 97% sequence identity, at least 98% sequence identity, at least 99% sequence identity, or at least 100% sequence identity to a sequence selected from: SEQ ID NO: 4 (S. pyogenes Dpr), SEQ ID NO: 5 (S. pyogenes Dpr), SEQ ID NO: 2 (GBS Ferritin KLL24083), SEQ ID NO: 6 (GBS 092), SEQ ID NO: 7 (GBS 092 with Cys to Ser modification), and SEQ ID NO: 8 (GBS 14747).

C2. The polypeptide according to C1, which is a naturally occurring S. agalactiae protein.

C3. A polypeptide capable of self-assembling into a nanoparticle, said polypeptide comprising or consisting of a naturally occurring S. agalactiae protein sequence where one or more naturally occurring cysteine residues have been substituted with serine.

C4. A polypeptide comprising or consisting of a sequence selected from: SEQ ID NO: 2 (GBS Ferritin KLL24083), SEQ ID NO: 6 (GBS 092), SEQ ID NO: 7 (GBS 092 with Cys to Ser modification), SEQ ID NO: 9 (GBS 092 with Cys to Ser modification and C-terminal his tag), SEQ ID NO: 10 (14747 + C-terminal His tag), SEQ ID NO: 11 (14747 + N-terminal helix), SEQ ID NO: 13 (14747 + N-terminal helix + C-term his tag), and SEQ ID NO: 18 (092 + N-term helix).

C5. The polypeptide according to any one of C1, C2, and C3, further comprising a purification tag, such as a histidine tag.

C6. The polypeptide according to C5, where said purification tag is selected from SEQ ID NOs: 19-23.

C7. The polypeptide according to C5, where said purification tag is not located N-terminally to said polypeptide.

C8. The polypeptide according to any one of C1, C2, and C3, further comprising a histidine tag located C-terminally to said polypeptide.

C9. The polypeptide according to any one of C1, C2, and C3, further comprising an amino acid sequence forming an alpha helix, said amino acid sequence located N-terminally to said polypeptide.

C10. The polypeptide according to C9, where said amino acid sequence comprises or consists of SEQ ID NO: 15.

C11. A polypeptide according to any one of C1, C2, C3 and C4, further comprising an N-terminal capture sequence.

C12. A polypeptide according to C1, further comprising an antigenic molecule at the N-terminus.

C13. A polypeptide according to C12, where said antigenic molecule is a polypeptide.

C14. A polypeptide according to C12, where said antigenic molecule is a polysaccharide or immunogenic fragment thereof.

C15. A polypeptide according to C12, where said antigenic molecule is a glycoconjugate.

C16. A polypeptide according to C14 where said polysaccharide is a bacterial capsular polysaccharide.

C17. A polypeptide according to C16 where said bacterial capsular polysaccharide is from Group B streptococcus.

C18. A polypeptide according to C12 where said polypeptide is a GBS surface protein or immunogenic fragment thereof.

C19. A polypeptide according to C11, further comprising a molecule bound to the capture sequence.

C20. A polypeptide according to C19, where said molecule bound to the capture sequence is a polypeptide or immunogenic fragment thereof.

C21. A polypeptide according to C19, where said molecule bound to the capture sequence is a polysaccharide or immunogenic fragment thereof.

C22. A polypeptide according to C19, where said molecule bound to the capture sequence is a glycoconjugate.

C23. A polypeptide according to C21 where said polysaccharide is a bacterial capsular polysaccharide.

C24. A polypeptide according to C23 where said bacterial capsular polysaccharide is from Group B streptococcus.

C25. A polypeptide according to C20 where said polypeptide is a GBS surface protein or immunogenic fragment thereof.

C26. An isolated nucleic acid molecule comprising a polynucleotide sequence encoding a polypeptide according to any one of C1-13, C18, C20, and C25.

C27. A recombinant vector comprising a nucleic acid molecule according to C26.

C28. A host cell comprising a vector according to C27.

C29. The host cell of C28, wherein the host cell is a mammalian cell, such as a Chinese Hamster Ovary (CHO) cell.

C30. The host cell of C28, wherein the host cell is a gram negative bacterial cell, such as Escherichia coli.

C31. A cell culture comprising the host cell of any one of C28-30.

C32. A process of producing a polypeptide comprising culturing the host cell of any one of C28-30 under suitable conditions in a suitable culture medium, thereby expressing the polypeptide encoded by the nucleic acid molecule carried by the vector.

C33. The process of C32, further comprising collecting expressed polypeptides from the cultured host cell(s) and/or culture medium, and optionally purifying the collected polypeptides.

C34. A polypeptide produced by the process of C32 or C33.

C35. A nanoparticle comprising one or more polypeptide subunits according any one of C1-25.

C36. The nanoparticle of C35, consisting of twelve polypeptide subunits having the same sequence.

C37. The nanoparticle of C35, further comprising one or more polypeptides bound to surface lysines of the polypeptide subunits.

C38. The nanoparticle of C37, where said polypeptides are bound to surface lysines of the polypeptide subunits.

C39. The nanoparticle of C37, where said polypeptide(s) are antigenic GBS surface proteins.

C40. The nanoparticle of C35, where at least one of said polypeptide subunits further comprises an antigenic molecule at the N-terminus.

C41. The nanoparticle of C35, where at least two, three, four, five, six, seven, eight, nine, ten, eleven or twelve of said polypeptide subunits further comprises an antigenic molecule at the N-terminus.

C42. An immunogenic composition comprising a polypeptide according to any one of C1-25, and/or a nanoparticle according to any one of C35-41.

C43. The immunogenic composition of C42, further comprising an adjuvant.

C44. The immunogenic composition of C43, wherein the adjuvant comprises an aluminum salt, a saponin, a liposaccharide, a lipopolysaccharide, an immunostimulatory nucleic acid molecule, a liposome, a Toll Receptor agonist, a water-in-oil emulsion, or an oil-in-water emulsion.

C45. A pharmaceutical composition comprising a pharmaceutically acceptable diluent, carrier or excipient polypeptide, and a polypeptide according to any one of C1-25, and/or a nanoparticle according to any one of C35-41.

C46. The pharmaceutical composition of C45, further comprising an adjuvant.

C47. The pharmaceutical composition of C46, wherein the adjuvant comprises an aluminum salt, a saponin, a liposaccharide, a lipopolysaccharide, an immunostimulatory nucleic acid molecule, a liposome, a Toll Receptor (TLR) agonist, a water-in-oil emulsion, or an oil-in-water emulsion.

C48. Use of a polypeptide according to any one of C1-25; a nanoparticle according to any one of C35-41; an immunogenic composition according to any one of C42-44; or pharmaceutical composition according to any one of C45-47; for the manufacture of a medicament for inducing an immune response.

C49. Use of a nanoparticle according to any one of C35-41; an immunogenic composition according to any one of C42-44; or pharmaceutical composition according to any one of C45-47; for inducing an immune response in a subject.

C50. Use of a nanoparticle according to any one of C35-41; an immunogenic composition according to any one of C42-44; or pharmaceutical composition according to any one of C45-47; in the prevention or treatment of disease.

C51. A nanoparticle according to any one of C35- 41 for use in the prevention or treatment of disease.

C52. A method of inducing an immune response in a subject, comprising administering to the subject an immunologically effective amount of a polypeptide according to any one of C1-25; nanoparticle according to any one of C35-41; an immunogenic composition according to any one of C42-44; or pharmaceutical composition according to any one of C45-47.

### Terms

To facilitate review of the various embodiments of this disclosure, the following explanations of terms are provided. Additional terms and explanations are provided in the context of this disclosure. Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

“Nanoparticles” as used herein refers to particles of less than about 100 nm in size (less than about 100 nm in maximum diameter for spherical, or roughly spherical, particles).

As used herein the terms “protein” and “polypeptide” are used interchangeably. A protein or polypeptide sequence refers to a contiguous sequence of two or more amino acids linked by a peptide bond. The proteins and polypeptides of the invention may comprise L-amino acids, D-amino acids, or a combination thereof.

As used herein, a “polypeptide subunit”, or “subunit”, refers to a polypeptide that, in combination with other polypeptide subunits, self-assembles into a nanoparticle. The subunit may comprise a polypeptide sequence which extends from the surface of the nanoparticle (i.e., is ‘displayed’ by the nanoparticle), a purification tag, or other modifications as are known in the art and that do not interfere with the ability to self-assemble into a nanoparticle.

As used herein, a “variant” polypeptide refers to a polypeptide having an amino acid sequence which is similar, but not identical to, a reference sequence, wherein the biological activity of the variant protein is not significantly altered. Such variations in sequence can be naturally occurring variations or they can be engineered through the use of genetic engineering techniques as known to those skilled in the art. Examples of such techniques may be found, e.g., in Sambrook et al., Molecular Cloning--A Laboratory Manual, 2nd Edition, Cold Spring HarborLaboratory Press, 1989, pp. 9.31-9.57), or in Current Protocols in Molecular Biology, John Wiley & Sons, N.Y. (1989), 6.3.1-6.3.6.

As used herein, a “fusion polypeptide” or “chimeric polypeptide” is a polypeptide comprising amino acid sequences from at least two unrelated proteins that have been joined together, via a peptide bond, to make a single polypeptide. The unrelated amino acid sequences can be joined directly to each other or they can be joined using a linker sequence. As used herein, polypeptides are unrelated if their amino acid sequences are not normally found joined together via a peptide bond in their natural environment(s) (e.g.,inside a cell). For example, the amino acid sequences of monomeric subunits that make up GBS ferritin, and the amino acid sequences of GBS surface proteins, are considered unrelated.

As used herein, an “antigen” is a molecule (such as a protein, saccharide, or glycoconjugate), a compound, composition, or substance that stimulates an immune response by producing antibodies and/or a T cell response in a mammal, including compositions that are injected, absorbed or otherwise introduced into a mammal. The term “antigen” includes all related antigenic epitopes. The term “epitope” or “antigenic determinant” refers to a site on an antigen to which B and/or T cells respond. The “predominant antigenic epitopes” are those epitopes to which a functionally significant host immune response, e.g., an antibody response or a T-cell response, is made. Thus, with respect to a protective immune response against a pathogen, the predominant antigenic epitopes are those antigenic moieties that when recognized by the host immune system result in protection from disease caused by the pathogen. The term “T-cell epitope” refers to an epitope that when bound to an appropriate MHC molecule is specifically bound by a T cell (via a T cell receptor). A “B-cell epitope” is an epitope that is specifically bound by an antibody (or B cell receptor molecule).

As used herein, the term “immunogenic” refers to the ability of a specific antigen, or a specific region thereof, to elicit an immune response to that antigen or region thereof when administered to a mammalian subject. The immune response may be humoral (mediated by antibodies) or cellular (mediated by cells of the immune system), or a combination thereof.

An “immune response” is a response of a cell of the immune system, such as a B cell, T cell, or monocyte, to a stimulus. An immune response can be a B cell response, which results in the production of specific antibodies, such as antigen specific neutralizing antibodies. An immune response can also be a T cell response, such as a CD4+ response or a CD8+ response. In some cases, the response is specific for a particular antigen (that is, an “antigen-specific response”), such as a GBS antigen. A “protective immune response” is an immune response that inhibits a detrimental function or activity of a pathogen, prevents infection by a pathogen in an individual, or decreases symptoms that result from infection by the pathogen. A protective immune response can be measured, for example, by measuring resistance to pathogen challenge in vivo.

The term “fragment,” in reference to a polypeptide (or polysaccharide) antigen, refers to a contiguous portion (that is, a subsequence) of that polypeptide (or polysaccharide). An “immunogenic fragment” of a polypeptide or polysaccharide refers to a fragment that retains at least one immunogenic epitope (e.g., a predominant immunogenic epitope or a neutralizing epitope); such fragments may be at least 10, 20, 30, 40, 50, 100 or more amino acids in length.

An “effective amount” means an amount sufficient to cause the referenced effect or outcome. An “effective amount” can be determined empirically and in a routine manner using known techniques in relation to the stated purpose. An “immunologically effective amount” is a quantity of an immunogenic composition sufficient to elicit an immune response in a subject (either in a single dose or in a series). Commonly, the desired result is the production of an antigen (e.g., pathogen)-specific immune response that is capable of or contributes to protecting the subject against the pathogen. Obtaining a protective immune response against a pathogen can require multiple administrations of the immunogenic composition. Thus, in the context of this disclosure, the term immunologically effective amount encompasses a fractional dose that, in combination with previous or subsequent administrations, induces a protective immune response.

As used herein, a “glycoconjugate” is a carbohydrate moiety (such as a polysaccharide) covalently linked to a moiety that is a different chemical species, such as a protein, peptide, lipid or lipid. Conjugation of polysaccharide antigens to suitable protein carriers is known in the art to increase immunogenicity of the polysaccharide antigen; such glycoconjugates are known for use in vaccination.

As used herein, where a nucleic acid sequence is operably linked to another polynucleotide molecule that it is not associated with in nature, the two sequences may be referred to as “heterologous” with regard to each other. Similarly, when a polypeptide is covalently linked to (including via a linker or intervening sequence), or is in a complex with, another protein that it is not associated with in nature, the polypeptides may be referred to as “heterologous” with regard to each other. A polypeptide (or nucleic acid) sequence that is “heterologous” to GBS refers to a polypeptide (or nucleic acid) sequence that is not found in naturally occurring GBS cells. Further, when a host cell comprises a nucleic acid molecule or polypeptide that it does not naturally comprise, the nucleic acid molecule and polypeptide may be referred to as “heterologous” to the host cell. For purposes of the present invention, in a fusion protein of two polypeptides from the same host organism (such as GBS), where the polypeptides are not naturally covalently associated with each other, the two polypeptides are considered heterologous to each other. Thus, for example, a protein comprising a GBS surface protein antigen attached to a GBS ferritin nanoparticle subunit would be considered a fusion protein of two heterologous polypeptide sequences.

“Operably linked” means connected so as to be operational, for example, in the configuration of recombinant polynucleotide sequences for protein expression. In certain embodiments, “operably linked” refers to the art-recognized positioning of nucleic acid components such that the intended function (e.g., expression) is achieved. A person with ordinary skill in the art will recognize that under certain circumstances, two or more components “operably linked” together are not necessarily adjacent to each other in the nucleic acid or amino acid sequence. A coding sequence that is “operably linked” to a control sequence (e.g., a promoter, enhancer, or IRES) is ligated in such a way that expression of the coding sequence is under the influence or control of the control sequence, but such a ligation is not limited to adjacent ligation.

By “adjacent”, it is meant “next to” or “side-by-side”. By “immediately adjacent”, it is meant adjacent to with no material structures in between (e.g., in the context of an amino acid sequence, two residues “immediately adjacent” to each other means there are atoms between the two residues sufficient to form the bonds necessary for a polypeptide sequence, but not a third amino acid residue).

By “c-terminally” or “c-terminal” to, it is meant toward the c-terminus. Therefore, by “c-terminally adjacent” it is meant “next to” and on the c-terminal side (i.e., on the right side if reading from left to right).

By “n-terminally” or “n-terminal” to, it is meant toward the n-terminus. Therefore, by “n-terminally adjacent” it is meant “next to” and on the n-terminal side (i.e., on the left side if reading from left to right).

As used herein, “mer” when referring to a protein nanoparticle, such as in means the number of subunit polypeptides that make up the NP. The subunit polypeptides do not have to be identical. Thus a 12-mer NP consists of twelve joined polypeptide subunits.

As used herein, a “recombinant” or “engineered” cell refers to a cell into which an exogenous DNA sequence, such as a cDNA sequence, has been introduced. A “host cell” is one that contains such an exogenous DNA sequence. “Recombinant” as used herein to describe a polynucleotide means a polynucleotide which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide.

A “subject” is a living multi-cellular vertebrate organism. In the context of this disclosure, the subject can be an experimental subject, such as a non-human mammal, e.g., a mouse, a rat, or a non-human primate. Alternatively, the subject can be a human subject.

The singular terms “a,” “an,” and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 200 pg, it is intended that the concentration be understood to be at least approximately (or “about” or “~”) 200 pg.

The term “comprises” means “includes.” Thus, unless the context requires otherwise, the word “comprises,” and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or groups thereof. The abbreviation, “e.g.” is used herein to indicate a non-limiting example and is synonymous with the term “for example.”

It is further to be understood that all base sizes or amino acid sizes, and all molecular weight or molecular mass values, given for nucleic acid molecules or polypeptides are approximate and are provided for description. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 200 pg, it is intended that the concentration be understood to be at least approximately (or “about” or “~”) 200 pg.

The term “and/or” as used in a phrase such as “A and/or B” is intended to include “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless specifically stated, a process comprising a step of mixing two or more components does not require any specific order of mixing. Thus components can be mixed in any order. Where there are three components then two components can be combined with each other, and then the combination may be combined with the third component, etc. Similarly, while steps of a method may be numbered (such as (1), (2), (3), etc. or (i), (ii), (iii)), the numbering of the steps does not mean that the steps must be performed in that order (i.e., step 1 then step 2 then step 3, etc.). The word “then” may be used to specify the order of a method’s steps.

The present invention is not limited to particular embodiments described herein. It is appreciated that certain features of the invention which are, for clarity, described in the context of separate embodiments, may also be provided in combination in a single embodiment. Conversely, various features of the invention which are, for brevity, described in the context of a single embodiment may also be provided separately or in any suitable sub-combination. All combinations of the embodiments are specifically embraced by the present invention and are disclosed hereinj ust as if each and every combination was individually and explicitly disclosed. In addition, all sub-combinations are also specifically embraced by the present invention and are disclosed herein just as if each and every such sub-combination was individually and explicitly disclosed herein.

Although methods and materials similar or equivalent to those described herein can be used in the practice or testing of this disclosure, suitable methods and materials are described below.

### EXAMPLES

### Example 1: GBS Lumazine Synthase

GBS Lumazine synthase (LS) was proposed as a potential antigen-carrying nanoparticle. The GBS LS subunit polypeptide as provided at GenBank Accession Q3K1V6 was modified to carry, at the N-terminal, two lysine residues and a His-Tag, as shown in SEQ ID NO:1. Expression of this polypeptide was attempted using both a bacterial and a mammalian system. However, in the bacterial expression system the LS was expressed in insoluble form. In the mammalian system, no expressed LS was detected.

GBS Lumazine Synthase Q3K1V6 + N-terminal lysine and His Tag - SEQ ID NO:1

KKGGSHHHHH HSGGMTIIEG QLVANEMKIG IVVSRFNELI TSKLLSGAVD
GLLRHGVSEE DIDIVWVPGA FEIPYMARKM ALYKDYDAII CLGVVIKGST
DHYDYVCNEV TKGIGHLNSQ SDIPHIFGVL TTDNIEQAIE RAGTKAGNKG
YDCALSAIEM VNLDKKLRER -170

### Example 2: Modified GBS Ferritin

The GBS ferritin subunit polypeptide as provided at GenBank Accession KLL24083.1 (SEQ ID NO:2) was modified by altering the cysteine residues at positions #4 and #124 to serine, as shown in SEQ ID NO:24. At the N-terminal, a double lysine and a His-tag were added, to provide SEQ ID NO:3.

GBS KLL24083.1 - SEQ ID NO:2

MKFCETKKIL NQLVADLSVF SVRIHQVHWY MRGGRFLTLH PKMDDLMEQI
NDQLDVISER LITLDGSPYS TLEEFFINSK LKEEKGSWNK TIDEQINYLL
EGYSYLISIY EEGIEIAGKE GDDCTEDIFI GSKSELEKEI WMLKAELGNS
PELDK - 155

Modified GBS KLL24083 (Cys to Ser at #4 and #124) - SEQ ID NO: 24

MKFS ETKKIL NQLVADLSVF SVRIHQVHWY MRGGRFLTLH PKMDDLMEQI
NDQLDVISER LITLDGSPYS TLEEFFINSK LKEEKGSWNK TIDEQINYLL
EGYSYLISIY EEGIEIAGKE GDDS TEDIFI GSKSELEKEI WMLKAELGNS
PELDK - 155

Modified GBS KLL24083.1 + N-terminal sequence - SEQ ID NO:3

KKGGSHHHHH HSGGMKFSET KKILNQLVAD LSVFSVRIHQ VHWYMRGGRF
LTLHPKMDDL MEQINDQLDV ISERLITLDG SPYSTLEEFF INSKLKEEKG
SWNKTIDEQI NYLLEGYSYL ISIYEEGIEI AGKEGDDSTE DIFIGSKSEL
EKEIWMLKAE LGNSPELDK - 169

SEQ ID NO:3 was expressed in a bacterial system. However, the purified polypeptides were not in the form of assembled nanoparticles.

### Example 3: Homology Modeling and Design - GBS “Ferritin-Like” Proteins

Bacterial “DNA-binding protein from starved cells (Dps)”-like proteins are involved in protecting the bacterial cell from oxidative stress. The naturally-occuring S. pyogenes DPS-like peroxide resistance protein (Dpr) nanoparticle is made up of twelve identical protein subunits assembled in a spherical structure with a hollow central cavity; this spherical molecule binds and oxidizes iron to prevent the formation of reactive oxygen species.

The Research Collaboratory for Structural Bioinformatics Protein Data Bank (RCSB PDB, accessible at www.wwPDB.org) identifies S. pyogenes Dpr as “2WLA”, and this identifier may be used herein. See also Haikarainen et al., Crystal Structures of S. Pyogenes Dpr Reveal a Dodecameric Iron-Binding Protein with a Ferroxidase Site. J Biol Inorg Chem 15(2):183-194 (February 2010).

Reported sequences of the polypeptide subunit of the S. pyogenes Dpr nanoparticle is provided at SEQ ID NO:4 (RCSB PDB sequence) and SEQ ID NO:5 (National Center for Biotechnology Information reference NP_269604.1). Different amino acid residues at position #79 are present in SEQ ID NOs: 4 and 5.

PDB 2WLA (S. pyogenes Dpr) - SEQ ID NO:4

MTNTLVENIY ASVTHNISKK EASKNEKTKA VLNQAVADLS VAASIVHQVH
WYMRGPGFLY LHPKMDELLD SLNANLDEV S ERLITIGGAP YSTLAEFSKH
SKLDEAKGTY DKTVAQHLAR LVEVYLYLSS LYQVGLDITD EEGDAGTNDL
FTAAKTEAEK TIWMLQAERG QGPAL - 175

NCBI Reference Sequence: NP_269604.1 - (S. pyogenes Dpr) - SEQ ID NO:5\

MTNTLVENIY ASVTHNISKK EASKNEKTKA VLNQAVADLS VAASIVHQVH
WYMRGPGFLY LHPKMDELLD SLNANLDEM S ERLITIGGAP YSTLAEFSKH
SKLDEAKGTY DKTVAQHLAR LVEVYLYLSS LYQVGLDITD EEGDAGTNDL
FTAAKTEAEK TIWMLQAERG QGPAL - 175

GBS proteins with homology to the S. pyogenes DPS-like peroxide resistance polypeptide subunit were identified by in silico database searches. One such protein was identified from a strain of GBS isolated from a human (GBS DK-PW-092) and one from a strain isolated from a bovine source (GBS LMG Strain 14747). The GBS DK-PW-092 sequence (GenBank KLL27267.1; SEQ ID NO:6) contained a Cysteine at residue 124; homology modeling suggested this cysteine would not establish an intramolecular or intra-nanoparticle disulfide bridge and so it was replaced with a Serine to avoid potential aggregation, to provide SEQ ID NO:7. Polypeptides of SEQ ID NO:7 may be referred to herein as “Modified DK-PW-092”, “modified 092”, or simply “092”. The sequence of GBS LMG Strain 14747 is provided at SEQ ID NO: 8. Polypeptides of SEQ ID NO:8 may be referred to herein as LMG 14747 or “14747”.

Modified GBS DK-PW-092- SEQ ID NO:7

MKFEKTKEIL NQLVADLSQF SVVIHQTHWY MRGPEFLTLH PQMDEYMDQI
NEQLDVVSER LITLDGSPFS TLREFAENTK IEDEIGNWDR TIPERMEKLV
AGYRYLADLY AKGIEVSGEE GDDSTQDIFI ANKTDIEKNI WMLQAKLGKA
PGIDA - 155

GBS LMG 14747 - SEQ ID NO: 8

MVYEKTQAVL NQAVADLSKA SSIVHQTHWY MRGPGFFYLH PKMDELMDEL
NETLDVVSER LITIGGAPYS TLKEFDEHSK LTEEVGSYDK SMTERLEKLA
DVYAYLSDLF QEGLDVSDEE GDDPSNGIFS DAQDAVQKTL WMLQAEIGRS
SGL -153

Constructs comprising SEQ ID NO: 7 or 8 and a C-terminal His tag attached by a flexible linker (GSSGHHHHHH (SEQ ID NO:19)), were produced; these sequences are provided as SEQ ID NOs: 9 and 10:

Modified GBS DK-PW-092 + C-terminal His tag - SEQ ID NO:9

MKFEKTKEIL NQLVADLSQF SVVIHQTHWY MRGPEFLTLH PQMDEYMDQI
NEQLDVVSER LITLDGSPFS TLREFAENTK IEDEIGNWDR TIPERMEKLV
AGYRYLADLY AKGIEVSGEE GDDSTQDIFI ANKTDIEKNI WMLQAKLGKA
PGIDAGSSGH HHHHH - 165

GBS LMG 14747 + C-terminal His tag - SEQ ID NO:10

MVYEKTQAVL NQAVADLSKA SSIVHQTHWY MRGPGFFYLH PKMDELMDEL
NETLDVVSER LITIGGAPYS TLKEFDEHSK LTEEVGSYDK SMTERLEKLA
DVYAYLSDLF QEGLDVSDEE GDDPSNGIFS DAQDAVQKTL WMLQAEIGRS
SGLGSSGHHH HHH - 163

The polypeptide subunit of S. pyogenes Dpr comprises an N-terminal helical portion, as shown in SEQ ID NOs: 4 and 5. A similar helical sequence was not present in the GBS 092 or 14747 sequences (SEQ ID NOs: 7 and 8). Because the absence of an N-terminal helical portion could affect the colloidal stability and yield of multimeric particles, a chimeric molecule was created where the first (N-terminal) three amino acids of SEQ ID NO:8 (14747) were replaced with the N-terminal 25 amino acids of S. pyogenes Dpr (SEQ ID NO:15), to provide a chimeric polyeptide (SEQ ID NO:11 “14747 + helix”); underlining indicates the N-terminal helix sequence.

GBS LMG 14747 + N-terminal helix - SEQ ID NO:11

MTNTLVENIY ASVTHNISKK EASKNEKTQA VLNQAVADLS KASSIVHQTH
WYMRGPGFFY LHPKMDELMD ELNETLDVVS ERLITIGGAP YSTLKEFDEH
SKLTEEVGSY DKSMTERLEK LADVYAYLSD LFQEGLDVSD EEGDDPSNGI
FSDAQDAVQK TLWMLQAEIG RSSGL - 175

Additionally, a construct was created where the first (N-terminal) three amino acids of the 14747 sequence (SEQ ID NO:8) were replaced with a lysine-rich trimeric helix (SEQ ID NO:16) and a T-cell PADRE epitope (SEQ ID NO:17). The resulting construct’s sequence is provided as SEQ ID NO:12 and contains (from N to C terminal) the 21 amino acid lysine-rich trimeric helix (underlined), the 13 amino acid T-cell PADRE epitope (double underlined), and amino acids 4-153 of SEQ ID NO:8. (Regarding the PADRE epitope, see Miller et al. PLoS One 9.12 (2014)).

GBS LMG 14747 + N-terminal Lysine-rich trimer - SEQ ID NO:12

MKKIEKRIEK IEKRIKKIEK RAKFVAAWTL KAAAEKTQAV LNQAVADLSK
ASSIVHQTHW YMRGPGFFYL HPKMDELMDE LNETLDVVSE RLITIGGAPY
STLKEFDEHS KLTEEVGSYD KSMTERLEKL ADVYAYLSDL FQEGLDVSDE
EGDDPSNGIF SDAQDAVQKT LWMLQAEIGR SSGL -184

These constructs were further modified to contain C-terminus His tags, as shown in SEQ ID NOs: 13 and 14:

GBS LMG 14747 + N-terminal helix + C-terminal His Tag - SEQ ID NO: 13

MTNTLVENIY ASVTHNISKK EASKNEKTQA VLNQAVADLS KASSIVHQTH
WYMRGPGFFY LHPKMDELMD ELNETLDVVS ERLITIGGAP YSTLKEFDEH
SKLTEEVGSY DKSMTERLEK LADVYAYLSD LFQEGLDVSD EEGDDPSNGI
FSDAQDAVQK TLWMLQAEIG RSSGLGSSGH HHHHH - 185

GBS LMG 14747 + N-terminal Lysine-rich trimer + C-terminal His Tag - SEQ ID NO:14

MKKIEKRIEK IEKRIKKIEK RAKFVAAWTL KAAAEKTQAV LNQAVADLSK
ASSIVHQTHW YMRGPGFFYL HPKMDELMDE LNETLDVVSE RLITIGGAPY
STLKEFDEHS KLTEEVGSYD KSMTERLEKL ADVYAYLSDL FQEGLDVSDE
EGDDPSNGIF SDAQDAVQKT LWMLQAEIGR SSGLGSSGHH HHHH -194

The expected Molecular Weight and theoretical isoelectric point (pI) of these polypeptides was calculated, as shown in Table 1.

TABLE 1

| SEQ ID NO: | Polypeptides | Expected MW* (monomer) (kDa) | Theoretical pl |
|---|---|---|---|
| SEQ ID NO:10 | GBS 14747 + His Tag | 18.3 | 4.74 |
| SEQ ID NO:9 | GBS 092 + His Tag | 19.0 | 5.00 |
| SEQ ID NO:13 | GBS 14747 + helix + His tag | 20.7 | 4.93 |
| SEQ ID NO:14 | GBS 14747 + Lysine-rich trimer + His tag | 22.0 | 5.68 |

* The determined MW of a polypeptide may vary depending upon the conditions used to assess its MW.

### Example 4: Expression and Purification of GBS Ferritin-Like Nanoparticles

Separate nucleic acid sequences encoding polypeptides of SEQ ID NOs: 9, 10, 13 and 14 were produced. Nucleic acid sequences were inserted into pET28b vector (Promega) using Gibson Assembling Kit (New England Bio), and polypeptides were expressed in E. coli (BL21(DE3) Star strain (Thermo Fisher Scientific)) using 1 mM IPTG fast induction at 37° C. for 4 hours. Polypeptides of SEQ ID NOs: 9, 10, 13 and 14 were produced in separate 100 mL E. coli bacterial cultures. The polypeptides naturally self-assembled into NPs.

Cell culture was harvested by centrifuging at 4,000 rpm for 20 minutes. From each culture, a cell pellet was collected and resuspended in 2 ml of 1X BUGBUSTER™ Detergent (Thermo Fisher Scientific), 20 mM Tris (tris(hydroxymethyl) aminomethane) buffer, pH 7.5, 100 mM NaCl, 1X protease cocktail, with 4 µl of DNAse and 4 µl of lysozyme for cell lysis. The resulting sample was centrifuged at 13000 rpm and filtered at 0.45 µm. The Kingfisher 96-Well Automated Protocol was used to purify the his-tag polypeptides via HISPUR™ Ni-NTA Magnetic Beads.

Boiled and reduced SDS-PAGE gels were performed on the eluted fractions. All four of the expressed polypeptides were soluble or partially soluble. Polypeptides of each of SEQ ID NOs: 9, 10, 13 or 14 exhibited varying oligomeric states on SDS Page.

Polypeptides of SEQ ID NO:14 (GBS 14747 + Lysine-rich trimer + His tag) were not detected in the elution fraction of the SDS gel.

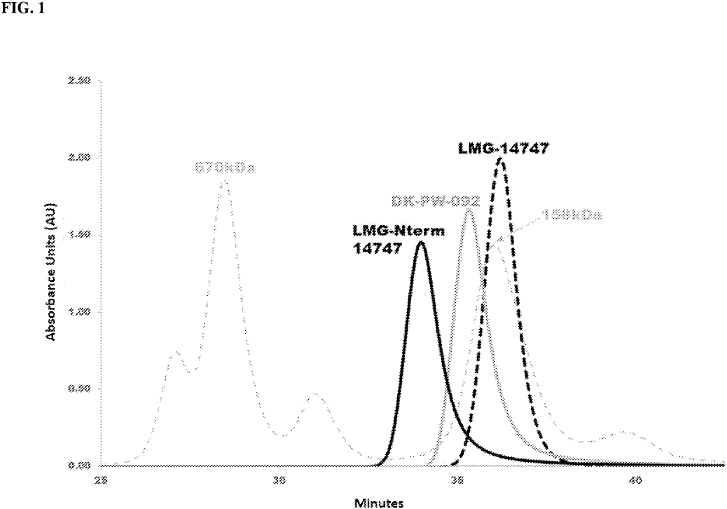

Purification: High Performance Liquid Chromatography-Size Exclusion Chromatography (HPLC-SEC) was performed on separate samples of NPs made up of subunits of either SEQ ID NO: 9, SEQ ID NO: 10, or SEQ ID NO:13, using an SEC-200 column (20 mM Tris, pH 7.5, 100 mM NaCl). Molecular weight distributions were similar to the theoretical size of dodecameric particles (FIG. 1). In FIG. 1, the solid black line represents SEQ ID NO:13 (GBS 14747 + N-terminal helix + His tag), the solid gray line represents SEQ ID NO:9 (GBS 092 + His Tag), the dashed black line represents SEQ ID NO:10 (GBS 14747 + His Tag), and the dashed gray line represents a 670 kDa molecular weight (MW) marker.

After confirming the expected MW, a larger batch of samples was purified with Fast Protein Liquid Chromatography-Size Exclusion Chromatography (FPLC-SEC).

Theoretical Molecular Weights of NPs were calculated, as shown in Table 2:

TABLE 2

| SEQ ID NO: | Polypeptide | Theoretical MW* (dodecamer)(kDa) |
|---|---|---|
| SEQ ID NO: 10 | GBS 14747 + His Tag | 219.6 |
| SEQ ID NO: 9 | GBS 092 + His Tag | 228 |
| SEQ ID NO: 13 | GBS 14747 + S. pyogenes helix + His tag | 248.4 |

*The determined MW of a polypeptide may vary depending upon the conditions used to assess its MW.

### Example 5: Characterization of GBS Ferritin-Like NPs

### Negative Stain Electron Microscopy

Samples of three different purified dodecameric nanoparticles were examined by negative stain Electron Microscopy (NS-EM). The nanoparticles comprised twelve subunits having the same sequence, selected from SEQ ID NO:9 (GBS 092 + His tag), SEQ ID NO:10, (GBS 14747 + His Tag), or SEQ ID NO:13 (GBS 14747 + helix + his tag). NS-EM grids were prepared by applying a sample of the nanoparticle onto a carbon coated copper grid, followed by staining with Methylamine Tungstate. Grids were then screened using a JEOL JEM-1230 Transmission Electron Microscope (TEM).

Negative stain micrographs showed homogeneously distributed nanoparticles for each of the three nanoparticles tested (micrographs not shown). Properly formed 12-mer nanoparticles were observed under NS-EM. The expected hydrodynamic diameter of the nanoparticles (homology model) is about 10 nanometers.

### Biophysical Studies

Polypeptides of SEQ ID NO: 9, SEQ ID NO: 10, and SEQ ID NO: 13 were produced by large scale purification (500 mL culture volume), and provided nanoparticles in the yields shown in TABLE 3.

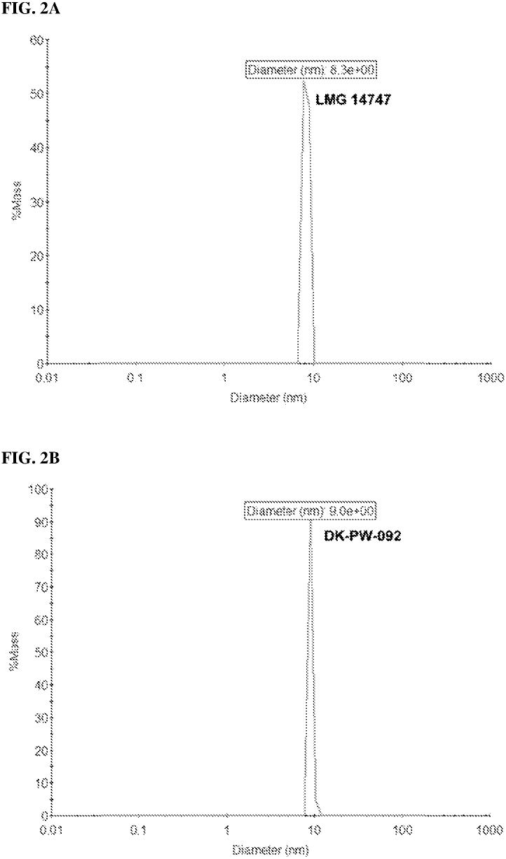

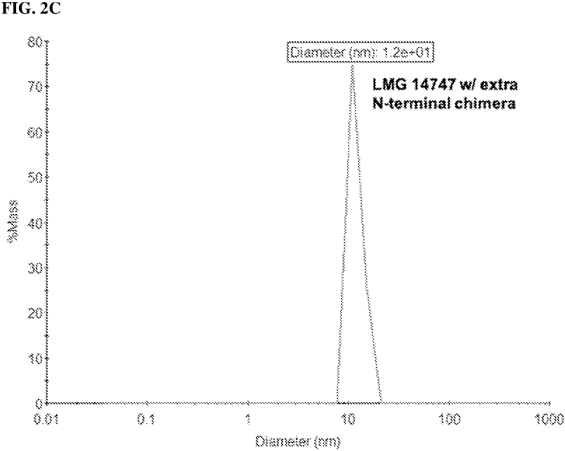

Dynamic Light Scattering: Samples of each nanoparticle were studied via Dynamic Light Scattering (DLS) to assess the size distribution profile. Results of DLS are shown in FIG. 2A (SEQ ID NO:10), FIG. 2B (SEQ ID NO:9), and FIG. 2C (SEQ ID NO:13), and establish that each of the three nanoparticles samples contained a monodisperse population, with nanoparticles of approximate diameter of 10 nanometers (consistent with NS-EM assessment discussed herein).

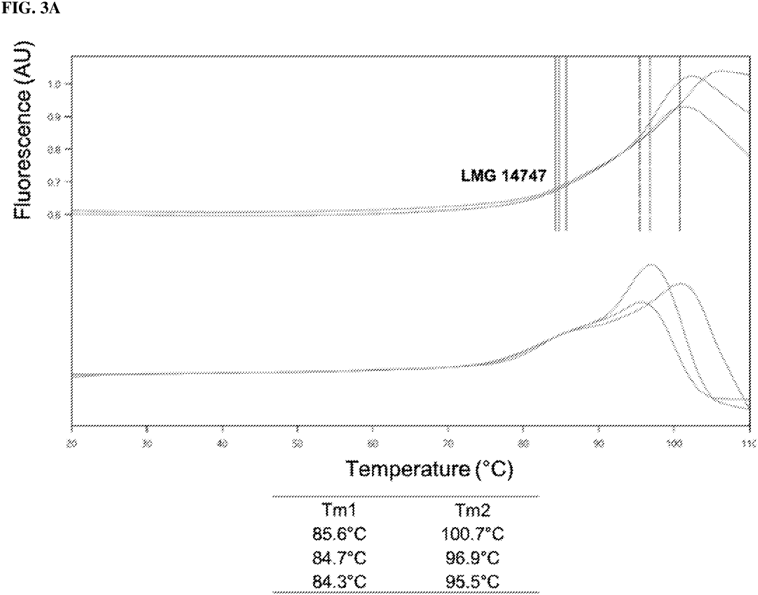

Samples of each nanoparticle were further studied via Nano-Differential Scanning Fluorimetry (Nano-DSF) to determine thermostability. Samples were run in triplicate. Results of Nano-DSF are shown in FIG. 3A (SEQ ID NO:10), FIG. 3B (SEQ ID NO:9), and FIG. 3C (SEQ ID NO:13). Average Tm values for each nanoparticle was at least about 80° C.

Biophysical characteristics of the NPs made up of SEQ ID NOs: 9, 10 and 13 are summarized in Table 3. These results indicate that the nanoparticles have monodisperse size distributions and have thermal transitions at about 80° C. or above.

TABLE 3

| | GBS 14747 + His Tag (SEQ ID NO:10) | GBS 092 + His Tag (SEQ ID NO:9) | GBS 14747 + helix + His Tag (SEQ ID NO:13) |
|---|---|---|---|
| Origin | Bovine | Homo Sapiens | Bovine |
| Thermostability (Nano-DSF) | Tm ≥ 80° | Tm ≥ 80° | Tm ≥ 80° |
| Morphology (NS-EM) | ~10 nm nanoparticle | ~10 nm nanoparticle | ~10 nm nanoparticle |
| Polydispersity (DLS) | Monodisperse | Monodisperse | Monodisperse |
| Yield from 500 ml culture | ~ 20 mg | ~100 mg | ~40 mg |

Morphology: diameter of nanoparticle is approximate.

### Example 6: X-ray Crystallography

Crystals of dodecameric nanoparticles made up of either SEQ ID NO: 9, 10, or 13 were flash frozen and shipped to the Advanced Photon Source (APS) for data collection. High resolution structures were determined for nanoparticles of SEQ ID NO:9 (1.9 Å) and SEQ ID NO:13 (1.8 Å). The structures were solved by molecular replacement from models based on the S. pyogenes Dpr sequence (SEQ ID NO:4) obtained using Swiss-Model. Each structure was found to contain bound metal atoms, presumed to be Fe+ atoms. The structures corresponded to dodecameric nanoparticles composed of twelve polypeptide subunits, with underlying tetrahedral symmetry.

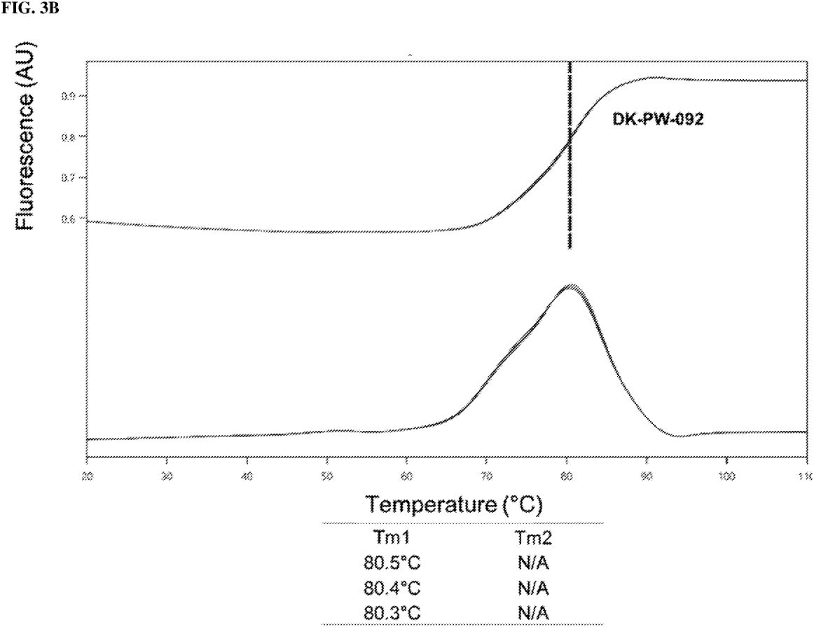

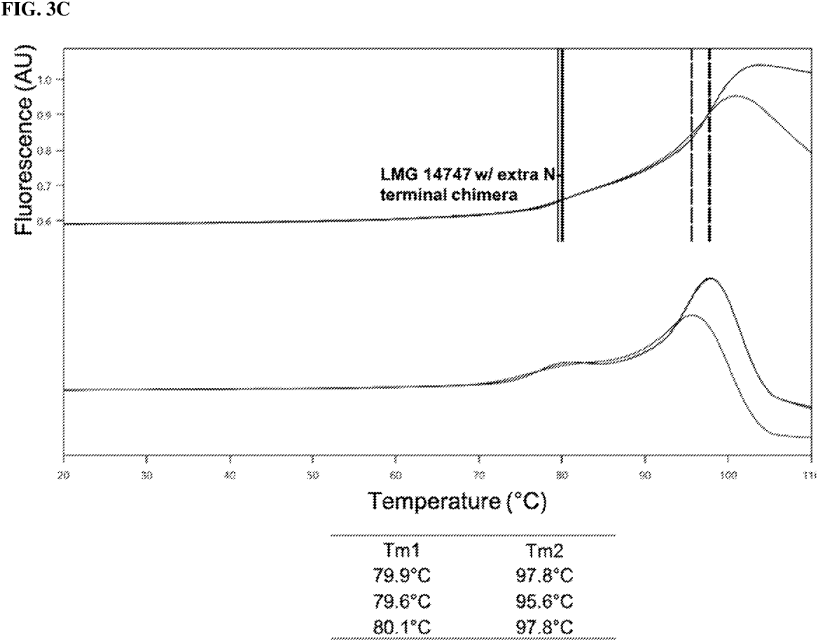

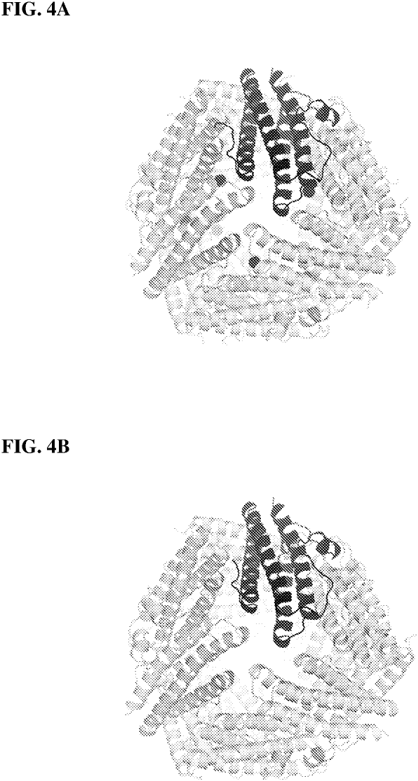

FIG. 4A graphically depicts the X-ray structure of the DK-PW-092+ His tag (SEQ ID NO: 9) nanoparticle. FIG. 4B graphically depicts the X-ray structure of LMG 14747 + helix + His tag (SEQ ID NO: 13). In FIGS. 4A and 4B, asymmetric units are in black, whereas biologically assembled nanoparticles are shown in grey. Metal particles are shown as black spheres.

Refinement statistics from the X-ray crystallography are provided in Table 4A (SEQ ID NO: 9) and Table 4B (SEQ ID NO: 13).

Purified protein samples were buffer exchanged into 10 mM Hepes pH 7.5, 150 mM NaCl, 5% glycerol and concentrated to 4 mg/mL. Four sparse matrix crystal screens were set up for each protein and stored in the Formulatrix Rock Imager at 25° C. After a week, multiple crystal hits for each protein were detected in the following conditions:
GBS LMG 14747 + His Tag (SEQ ID NO:10) : 0.1 M magnesium acetate, 0.1 M MES (2-(N-morpholino)ethanesulfonic acid) pH 6.5, 10 % w/v PEG (polyethylene glycol) 10000;
GBS 092 + His Tag (SEQ ID NO: 9): 0.4 M Ammonium phosphate monobasic; 0.2 M Magnesium acetate tetrahydrate, 0.1 M Sodium cacodylate trihydrate pH 6.5, 30% v/v (+/-)-2-Methyl- 2,4-pentanediol; 0.1 M Sodium acetate 4.6 8 % w/v PEG 4000; and
GBS LMG 14747 + Helix + His Tag (SEQ ID NO:13): 0.2 M sodium acetate 0.1 M sodium citrate 5.5 10 % w/v PEG 4000; 0.1 M sodium citrate 5.0 8 % w/v PEG 8000

TABLE 4A

Nanoparticles of SEQ ID NO: 9

| | |
|---|---|
| Resolution Range | 32.41 - 1.902 (1.97 - 1.902) |
| Space group | R3:H |
| Unit Cell | 95.789 95.789 207.733 90 90 120 |
| Total Reflections | 323853 (32633) |
| Unique Reflections | 55758 (5428) |
| Multiplicity | 5.8 (5.9) |
| Completeness (%) | 99.51 (97.26) |
| Mean I/sigma (I) | 20.55 (3.70) |
| Wilson B-factor | 19.34 |
| R-merge | 0.16 (0.8236) |
| R-meas | 0.1756 (0.9047) |
| R-pim | 0.07167 (0.3712) |
| CC½ | 0.991 (0.792) |
| CC* | 0.998 (0.94) |
| Reflections used in refinement | 55576 (5428) |
| Reflections used for R-free | 2004 (197) |
| R-work | 0.1582 (0.2192) |
| R-free | 0.1950 (0.2854) |
| CC(work) | 0.959 (0.778) |
| CC(free) | 0.940 (0.779) |
| Number of non-hydrogen atoms | 5791 |
| Macromolecules | 4987 |
| ligands | 4 |
| Solvent | 800 |
| Protein residues | 615 |
| RMS (bonds) | 0.010 |
| RMS (angles) | 1.41 |
| Ramachandran favored (%) | 98.52 |
| Ramachandran allowed (%) | 1.48 |
| Ramachandran outliers (%) | 0.00 |
| Rotamer outliers (%) | 0.18 |
| Clashscore | 4.47 |
| Average B-factor | 20.71 |
| Macromolecules | 18.28 |
| Ligands | 25.98 |
| solvent | 35. |

TABLE 4B

Nanoparticles of SEQ ID NO:13

| | |
|---|---|
| Resolution Range | 31.69 - 1.8 (1.865 - 1.8) |
| Space group | F41 3 2 |
| Unit Cell | 187.497 187.497 187.497 90 90 90 |
| Total Reflections | 1080004 (110709) |
| Unique Reflections | 26697 (2608) |
| Multiplicity | 40.5 (42.4) |
| Completeness (%) | 99.93 (99.92) |
| Mean I/sigma (I) | 28.03 (1.87) |
| Wilson B-factor | 17.86 |
| R-merge | 0.1737 (2.395) |
| R-meas | 0.1759 (2.424) |
| R-pim | 0.02752 (0.3701) |
| CC½ | 0.999 (0.849) |
| CC* | 1 (0.958) |
| Reflections used in refinement | 26689 (2608) |
| Reflections used for R-free | 1988 (194) |
| R-work | 0.1618 (0.2300) |
| R-free | 0.1833 (0.2670) |
| CC(work) | 0.963 (0.901) |
| CC(free) | 0.964 (0.896) |
| Number of non-hydrogen atoms | 1400 |
| Macromolecules | 1203 |
| Solvent | 197 |
| Protein residues | 152 |
| RMS (bonds) | 0.008 |
| RMS (angles) | 0.90 |
| Ramachandran favored (%) | 98.67 |
| Ramachandran allowed (%) | 1.33 |
| Ramachandran outliers (%) | 0.00 |
| Rotamer outliers (%) | 0.00 |
| Clashscore | 0.42 |
| Average B-factor | 21.63 |
| Macromolecules | 19.55 |
| solvent | 34.35 |

### Example 7: Lysine Chemical Conjugation Sites

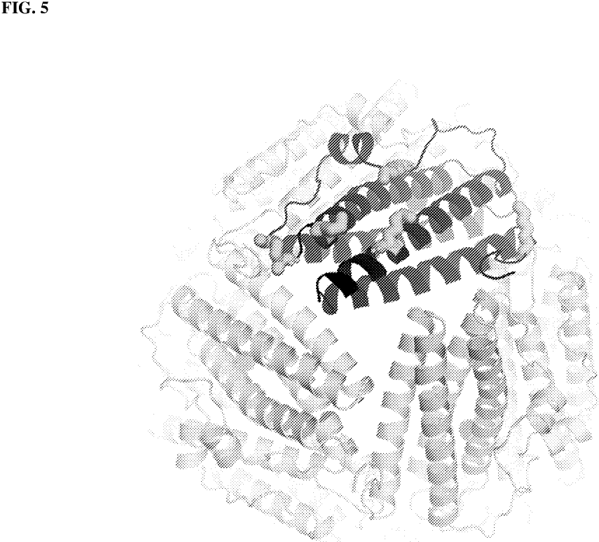

Surface-exposed lysine residues for use in conjugating glycans to the nanoparticles were identified in the nanoparticle made of GBS 092 + His Tag (SEQ ID NO:9), which contains ten lysine residues. FIG. 5 shows 1.9 Angstrom crystal structure of the dodecameric NP made up of subunits of SEQ ID NO:9, with exposed lysines highlighted as sticks. TABLE 5 lists the lysine residues (position numbering according to SEQ ID NO:9), with exposed lysines indicated by bolding and underlining, and also provides the relative exposure (% exposure) and Solvent Accessible Surface Area (SASA) of each lysine residue.

TABLE 5

| Lysine # (SEQ ID NO:9) | SASA (Angstrom²) | % Exposure |
|---|---|---|
| 2 | 156.1 | 66.3 |
| 5 | 113.1 | 48.0 |
| 7 | 26.3 | 11.2 |
| 80 | 146.8 | 62.3 |
| 98 | 60.4 | 25.7 |
| 112 | 97.4 | 41.4 |
| 133 | 22.1 | 9.4 |
| 138 | 84.1 | 35.7 |
| 146 | 1.3 | 0.5 |
| 149 | 95.4 | 40.5 |

MK FEK TKEIL NQLVADLSQF SVVIHQTHWY MRGPEFLTLH PQMDEYMDQI
NEQLDVVSER LITLDGSPFS TLREFAENTK IEDEIGNWDR TIPERMEKLV
AGYRYLADLY AK GIEVSGEE GDDSTQDIFI ANKTDIEKNI WMLQAKLGK A
PGIDAGSSGH HHHHH - 165 (SEQ ID NO: 9)

### Example 8: Production of Short GBS Capsular Oligosaccharides and Conjugation to NP

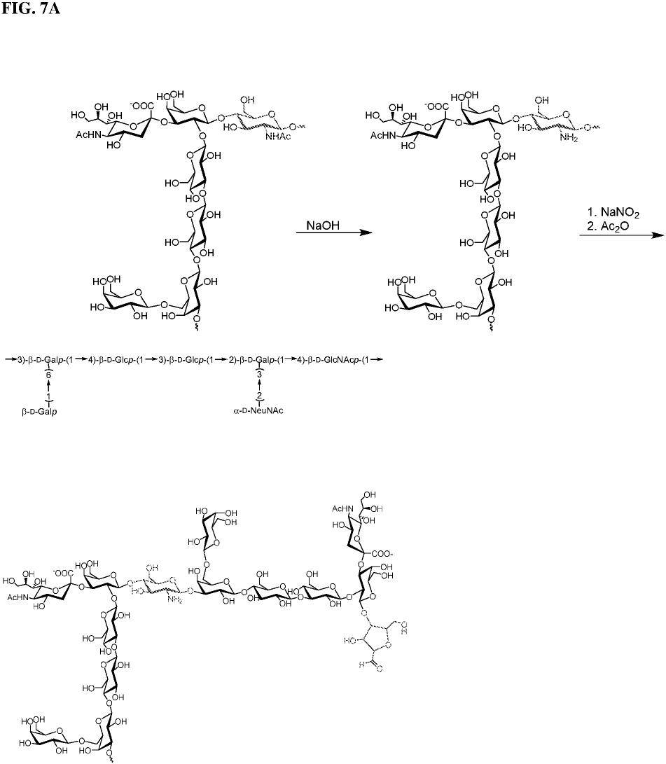

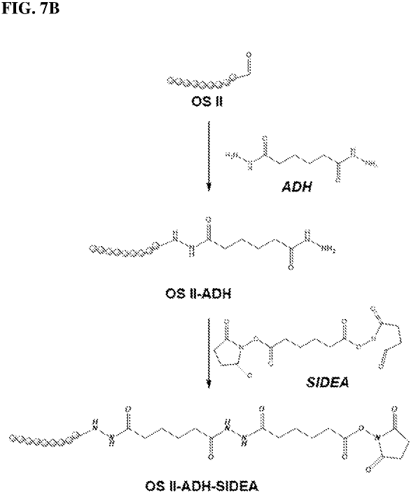

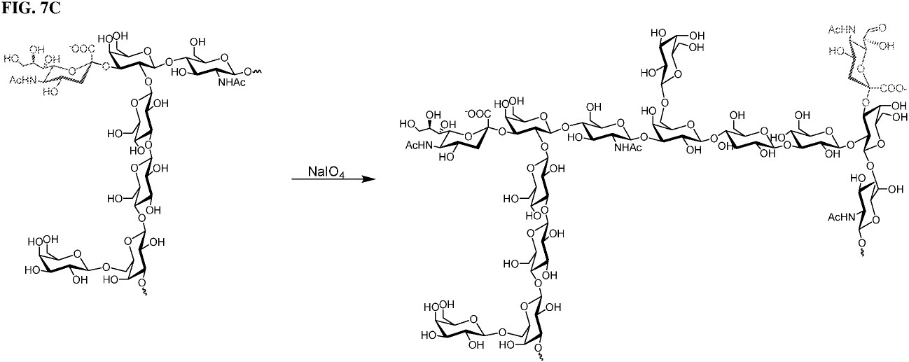

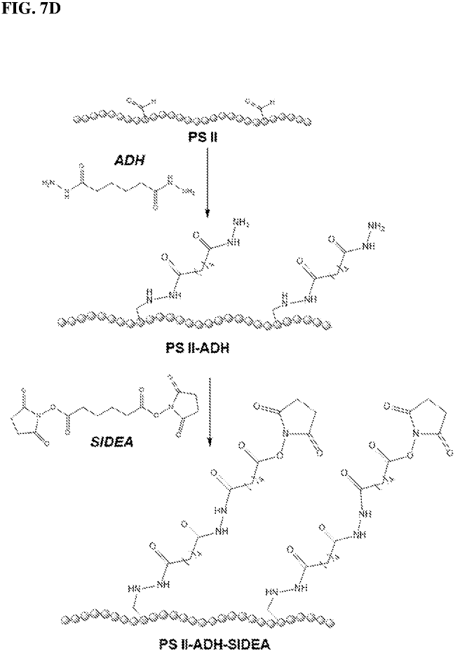

Short oligosaccharides: GBS serotype II capsular polysaccharides were depolymerized to provide shorter oligosaccharides by: N-deAcetylation with NaOH, oxidation with NaNO₂, and N-acetylation, followed by purification using a desalting column, as shown in FIG. 7A. The resulting oligosaccharides were then separated using anionic exchange chromatography, and different gradient elution to separate the different sizes of oligosaccharides. Identity and structural conformity of the GBS CPS II oligosaccharides were assessed by 1H NMR. Size was analyzed using HPLC-SEC, and saccharide content assessed using HPAEC-PAD or Colorimetric assay (NeuNAc-based).

The GBS serotype II short oligosaccharides were modified with a hydrazine linker (ADH) and an active ester spacer (SIDEA), as shown in FIG. 7B. These modified oligosaccharides are then conjugated to GBS Ferritin NPs of the present invention, such as by contacting the oligosaccharides and Nanoparticles at 15:1 (mol/mol), at room temperature, for approximately 12 hours.

### Example 9: Conjugation of GBS Capsular Polysaccharide to NP

Oxidation of GBS serotype II capsular polysaccharide was carried out using NaIO₄, as shown in FIG. 7C. The resulting polysaccharides were purified using a desalting column. Identity and structural conformity of the resulting polysaccharides were assessed by 1H NMR. Saccharide content assessed using HPAEC-PAD or Colorimetric assay (NeuNAc-based).

The GBS serotype II CPS were modified with a hydrazine linker (ADH) and an active ester spacer (SIDEA), as shown in FIG. 7D. These modified polysaccharides are then conjugated to GBS Ferritin NPs of the present invention, such as by contacting the oligosaccharides and Nanoparticles at 15:1 (mol/mol), at room temperature, for approximately 12 hours.

We claim:

1. A polypeptide capable of self-assembling into a nanoparticle, the polypeptide comprising of a sequence having at least 80% sequence identity, at least 85% sequence identity, at least 87% sequence identity, at least 90% sequence identity, at least 92% sequence identity, at least 94% sequence identity, at least 95% sequence identity, at least 96% sequence identity, at least 97% sequence identity, at least 98% sequence identity, at least 99% sequence identity, or 100% sequence identity to a sequence selected from: SEQ ID NO: 4, SEQ ID NO: 5, SEQ ID NO: 2, SEQ ID NO: 6, SEQ ID NO: 7, and SEQ ID NO: 8.

2. (canceled)

3. The polypeptide of claim 1, wherein the polypeptide comprises a S. agalactiae protein sequence where one or more naturally occurring cysteine residues have been substituted with serine.

4. A polypeptide comprising of a sequence selected from: SEQ ID NO: 2, SEQ ID NO: 6, SEQ ID NO: 7, SEQ ID NO: 9, SEQ ID NO: 10, SEQ ID NO: 11, SEQ ID NO: 13, and SEQ ID NO: 18.

5. The polypeptide according to claim 1 further comprising a purification tag, wherein the purification tag is not located N-terminally to the polypeptide.

6. The polypeptide according to claim 5, wherein the purification tag is selected from SEQ ID NOs: 19-23.

7. (canceled)

8. The polypeptide according to claim 5, further comprising a histidine tag located C-terminally to the polypeptide.

9. The polypeptide according to claim 1 further comprising an amino acid sequence forming an alpha helix, wherein the amino acid sequence is located N-terminally to the polypeptide.

10. The polypeptide according to claim 9, wherein the amino acid sequence comprises SEQ ID NO: 15.

11. The polypeptide according to claim 1, further comprising an N-terminal capture sequence.

12. The polypeptide according to claim 1, further comprising an antigenic molecule at the N-terminus, wherein the antigenic molecule is (a) a polypeptide, (b) a polysaccharide or immunogenic fragment thereof, or (c) a glycoconjugate.

13-15. (canceled)

16. The polypeptide according to claim 12, wherein the antigenic molecule is a polysaccharide or immunogenic fragment thereof, wherein the polysaccharide is a bacterial capsular polysaccharide, and wherein the bacterial capsular polysaccharide is from Group B streptococcus.

17-18. (canceled)

19. The polypeptide according to claim 11, further comprising a molecule bound to the capture sequence.

20-25. (canceled)

26. An isolated nucleic acid molecule comprising a polynucleotide sequence encoding the polypeptide according to claim 1.

27. A recombinant vector comprising the nucleic acid molecule of a claim 26.

28-34. (canceled)

35. A nanoparticle comprising one or more polypeptides according to claim 1.

36. (canceled)

37. The nanoparticle of claim 35, further comprising one or more polypeptides bound to surface lysines of the polypeptides wherein the polypeptides are antigenic GBS surface proteins.

38-41. (canceled)

42. An immunogenic composition comprising the polypeptide of claim 1.

43-44. (canceled)

45. A pharmaceutical composition comprising a pharmaceutically acceptable diluent, carrier or excipient polypeptide, and a to the polypeptide of claim 1.

46. The pharmaceutical composition of claim 45, further comprising an adjuvant, wherein the adjuvant comprises an aluminum salt, a saponin, a liposaccharide, a lipopolysaccharide, an immunostimulatory nucleic acid molecule, a liposome, a Toll Receptor (TLR) agonist, a water-in-oil emulsion, or an oil-in-water emulsion.

47-51. (canceled)

52. A method of inducing an immune response in a subject, comprising administering to the subject an immunologically effective amount of the polypeptide according to claim 1.
